(19) United States
(12) Patent Application Publication
(10) Pub. No.: US 2017/0106013 A1
(43) Pub. Date: Apr. 20, 2017

(54) BIOPHOTONIC HYDROGELS

(71) Applicant: KLOX Technologies Inc., Laval (CA)

(72) Inventors: Remigio Piergallini, Grottammare (IT); Nikolaos Loupis, Athens (GR); Joanna Jaworska, Montreal (CA); Emmanuelle Devemy, Montreal (CA); Éric DesRosiers, Outremont (CA); Abdellatif Chenite, Kirkland (CA)

(21) Appl. No.: 15/316,111

(22) PCT Filed: Jun. 4, 2015
(86) PCT No.: PCT/CA2015/050518

§ 371 Date: Dec. 2, 2016

Related U.S. Application Data
(60) Provisional application No. 62/012,770, filed on Jun. 16, 2014.
(60) Provisional application No. 62/007,911, filed on Jun. 4, 2014.

(51) Int. Cl. A61K 31/785; A61K 8/88; A61K 8/81; A61Q 19/08; A61K 41/00; A61N 5/06; C08J 3/075; C08K 5/1545; C08K 3/36; C08K 3/34; C08L 33/26; A61K 8/04; A61L 26/00

(52) U.S. Cl. CPC A61K 31/785; A61K 8/042; A61K 8/88; A61K 8/8158; A61Q 19/08; A61K 41/00; A61L 26/0019; A61L 26/0066; A61L 26/008; C08J 3/075; C08K 5/1545; C08K 3/36; C08K 3/34; C08L 33/26; A61N 5/062; A61K 2800/434; A61K 2800/48; A61K 2800/81; A61K 2800/95; A61L 2300/404; A61L 2300/442; C08J 2333/26

(57) ABSTRACT

The present disclosure provides biophotonic hydrogels and methods useful in phototherapy. In particular, the biophotonic hydrogels of the present disclosure include N-Hydroxyethyl acrylamide (HEAA), and at least one chromophore, wherein the at least one chromophore is not fully photobleached after photopolymerization. The biophotonic hydrogels and the methods of the present disclosure are useful for promoting wound healing and skin rejuvenation, as well as treating acne and various other skin disorders.

### FIELD OF THE DISCLOSURE

The present disclosure generally relates to forming biophotonic hydrogels.

### BACKGROUND OF THE DISCLOSURE

Phototherapy has recently been recognized as having wide range of applications in both the medical and cosmetic fields including use in surgery, therapy and diagnostics. For example, phototherapy has been used to treat cancers and tumors with lessened invasiveness, to disinfect target sites as an antimicrobial treatment, to promote wound healing, and for facial skin rejuvenation.

Hydrogels are materials which absorb solvents (such as water), undergo rapid swelling without discernible dissolution, and maintain three-dimensional networks capable of reversible deformation. Forming hydrogels has been proposed for use in a number of applications, including surgery, medical diagnosis and treatment, adhesives and sealers. One method for formation of hydrogels employs photopolymerization. Photopolymerization comprises using light to convert initiator molecules into free radicals that can react with monomers or macromers containing double bond and propagate radical chain polymerization. Forming hydrogels intended for biomedical and tissue engineering applications should occur under mild conditions, for example neutral pH and require non-toxic photoinitiators.

Therefore, it is an object of the present disclosure to provide new and improved formation of hydrogel compositions and methods that are useful in phototherapy.

### SUMMARY OF THE DISCLOSURE

The present disclosure provides biophotonic hydrogels and methods useful in phototherapy. In particular, biophotonic hydrogels of the present disclosure include a polymerisable monomer, and at least one chromophore. Preferably, the at least one chromophore can absorb and/or emit light to initiate photopolymerization of the hydrogel, and further wherein the at least one chromophore is not fully photobleached after photopolymerization.

In some embodiments, the biophotonic hydrogel composition further comprises a cross linker. In some embodiments, the cross linker is Poly(ethylene glycol) diacrylate (PEGDA).

The composition may also include an initiator. The initiator may be TEA. The composition may also include a catalyst, and the catalyst may be 1-vinyl-2 pyrrolidinone (NVP).

In some embodiments, the catalyst may be polyvinyl pyrrolidone (PVP).

In some embodiments, the chromophore absorbs and/or emits visible light. In some embodiments, the chromophore absorbs and/or emits light within the range of about 400 nm-750 nm or about 400-700 nm or about 400 nm-800 nm.

In some embodiments, the hydrogel composition further comprises a surfactant. In some embodiments, the surfactant is Pluronic F127. The surfactant may be present in the biophotonic hydrogel at between about 1-5 wt %, between about 2.5-7.5 wt %, between about 5-10 wt %, between about 7.5-12.5 wt %, between about 10-15 wt %, between about 12.5-17.5 wt %, between about 15-20 wt %, between about 20-25 wt % Pluronic® F127. In certain embodiments, the biophotonic hydrogel comprises a further surfactant comprising a cationic surfactant. In certain other embodiments, the cationic surfactant is cetyltrimethyl ammonium bromide (CTAB). In certain embodiments, the CTAB may be present in the biophotonic hydrogel at a percentage concentration to allow for a formation of micelles by the CTAB (termed a critical micelle concentration). In certain embodiments, the critical micelle concentration may be increased with an increase in incubation temperature of the biophotonic hydrogel.

In some embodiments, the hydrogel composition further includes a stabilizer. The stabilizer may be gelatin, hydroxyethyl cellulose (HEC), carboxymethyl cellulose (CMC) or any other thickening agent.

The chromophore of the present hydrogel composition may be a xanthene dye. The xanthene dye may be fluorescein or eosin, or any other xanthene dye.

In some embodiments, the biophotonic hydrogel composition further comprises an additional compound that may enhance the mechanical strength of the biophotonic hydrogel. In some embodiments, the additional compound may be a silica-based compound. In certain embodiments, the silica-based compound may be a silica clay or fumed silica (SiO₂). In certain embodiments, the silica clay may be bentonite. The bentonite may be present in the biophotonic hydrogel at between about 0.01-0.5 wt %, between about 0.25-0.75 wt %, between about 0.5-0.75 wt %, between about 0.75-1.0 wt % of the biophotonic hydrogel. The fumed silica may be present in the biophotonic hydrogel at between about 0.01-1.0 wt %, between about 1.0-2.0 wt %, between about 2.0-3.0 wt %, between about 3.0-4.0 wt %, between about 4.0-5.0 wt % of the biophotonic hydrogel.

In certain other embodiments, the biophotonic hydrogel comprises a combination of the further surfactant and the additional compound for enhancing the mechanical strength of the biophotonic hydrogel. In certain other embodiments, the combination of the further surfactant and the additional compound for enhancing the mechanical strength in the biophotonic hydrogel comprises CTAB and fumed silica, respectively.

The biophotonic hydrogel composition of any aspects or embodiments of the disclosure may be used for modulating a pro-inflammatory response in a cell or tissue type. In some embodiments, the biophotonic hydrogel composition of any aspects or embodiments of the disclosure may be used for stimulating an increase in collagen production in a cell, or tissue type, and in some embodiments, the biophotonic hydrogel composition of any aspects or embodiments of the disclosure may be used for stimulating fibroblast proliferation.

The biophotonic hydrogel composition of any aspects or embodiments of the disclosure may be used for cosmetic or medical treatment of tissue. In some embodiments, the cosmetic treatment is skin rejuvenation and conditioning, and the medical treatment is wound healing, periodontal treatment or acne treatment or treatment of other skin conditions including acne, eczema, psoriasis or dermatitis. In some aspects, the biophotonic hydrogel composition is used for modulating inflammation, modulating collagen synthesis or for promoting angiogenesis.

The present disclosure also provides methods for promoting wound healing comprising applying a biophotonic hydrogel composition over a wound, wherein the hydrogel composition comprises N-Hydroxyethyl acrylamide (HEAA) and at least one chromophore; and illuminating said biophotonic hydrogel composition with light having a wavelength that is absorbed by the at least one chromophore; wherein said method promotes wound healing.

The present disclosure also provides methods for treating a skin disorder, wherein the method comprises applying a biophotonic hydrogel composition over a target skin tissue, wherein the hydrogel composition comprises N-Hydroxyethyl acrylamide (HEAA), and at least one chromophore; and illuminating said biophotonic hydrogel composition with light having a wavelength that is absorbed by the at least one chromophore; and wherein said method promotes healing of said skin disorder. In some embodiments, the skin disorder is selected from acne, eczema, proriasis and dermatitis.

The present disclosure also provides methods for treating acne comprising: applying a biophotonic hydrogel composition over a target skin tissue, wherein the hydrogel composition comprises N-Hydroxyethyl acrylamide (HEAA), and at least one chromophore; and illuminating said biophotonic hydrogel composition with light having a wavelength that is absorbed by the at least one chromophore; and wherein said method treats the acne.

The present disclosure also provides methods for skin rejuvenation comprising applying a biophotonic hydrogel composition over a target skin tissue, wherein the hydrogel composition comprises N-Hydroxyethyl acrylamide (HEAA), and at least one chromophore; and illuminating said biophotonic hydrogel composition with light having a wavelength that is absorbed by the at least one chromophore; and wherein said method promotes skin rejuvenation.

The present disclosure also provides methods for preventing or treating scars comprising applying a biophotonic hydrogel composition over a target skin tissue, wherein the hydrogel comprises N-Hydroxyethyl acrylamide (HEAA), and at least one chromophore; and illuminating said biophotonic hydrogel composition with light having a wavelength that is absorbed the at least one chromophore; and wherein said method promotes prevents or treats scars.

### BRIEF DESCRIPTION OF THE DRAWINGS

Further aspects and advantages of the present disclosure will become better understood with reference to the description in association with the following in which:

FIG. 1 illustrates the light emission spectra of biophotonic poly(hydroxyethyl acrylamide) during 0-5 minutes of illumination, according to an embodiment of the present disclosure.

FIG. 2 illustrates the light emission spectra of biophotonic poly(hydroxyethyl acrylamide) during 5-10 minutes of illumination, according to an embodiment of the present disclosure.

FIG. 3 illustrates the light emission spectra of biophotonic poly(hydroxyethyl acrylamide)/gelatin during 0-5 minutes of illumination, according to an embodiment of the present disclosure.

FIG. 4 illustrates the light emission spectra of biophotonic poly(hydroxyethyl acrylamide)/gelatin during 5-10 minutes of illumination, according to an embodiment of the present disclosure.

FIG. 5 illustrates the light emission spectra of biophotonic poly(hydroxyethyl acrylamide)/HEC during 0-5 minutes of illumination, according to an embodiment of the present disclosure.

FIG. 6 illustrates the light emission spectra of biophotonic poly(hydroxyethyl acrylamide)/HEC during 5-10 minutes of illumination, according to an embodiment of the present disclosure.

FIG. 7 illustrates the light emission spectra of biophotonic poly(hydroxyethyl acrylamide)/Pl-F127 during 0-5 minutes of illumination, according to an embodiment of the present disclosure.

FIG. 8 illustrates the light emission spectra of biophotonic poly(hydroxyethyl acrylamide)/Pl-F127-CTAB during 0-5 minutes of illumination, according to an embodiment of the present disclosure.

FIG. 9 illustrates the light emission spectra of biophotonic poly(hydroxyethyl acrylamide)/Pl-F127-Bentonite during 0-5 minutes of illumination, according to an embodiment of the present disclosure.

FIG. 10 illustrates the light emission spectra of biophotonic poly(hydroxyethyl acrylamide)/Pl-F127-SiO₂ during 0-5 minutes of illumination, according to an embodiment of the present disclosure.

FIG. 11 illustrates the light emission spectra of biophotonic poly(hydroxyethyl acrylamide)/Pl-F127-SiO₂—CTAB during 0-5 minutes of illumination, according to an embodiment of the present disclosure.

FIG. 12 illustrates a graph indicating the modulation of collagen production in Human Dermal Fibroblasts (DHF) 48 hours after treatment with light from a blue light and a membrane according to one embodiment of the present disclosure.

FIG. 13 illustrates a graph indicating the modulation of Human Dermal Fibroblasts (DHF) proliferation 24 hours after treatment with light from a blue light and a membrane according to one embodiment of the present disclosure.

### DETAILED DESCRIPTION

### (1) Overview

The present disclosure provides biophotonic hydrogels and uses thereof. Biophotonic therapy using these materials would combine the beneficial effects of forming hydrogels with the photobiostimulation induced by the fluorescent light generated upon illumination of the materials. In certain embodiments of forming biophotonic hydrogels of the present disclosure are activated by visible light. Furthermore, in certain embodiments, phototherapy using the biophotonic hydrogels of the present disclosure will for instance promote wound healing, rejuvenate the skin by, e.g., promoting collagen synthesis, treat skin conditions such as acne, and treat periodontitis.

### (2) Definitions

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

“Biophotonic” means the generation, manipulation, detection and application of photons in a biologically relevant context. In other words, biophotonic compositions and materials exert their physiological effects primarily due to the generation and manipulation of photons.

“Hydrogel” refers to a material of solid or semi-solid texture that includes water. Hydrogels are formed by a three-dimensional network of molecular structures within which water, among other substances, may be held. The three-dimensional molecular network may be held together by covalent chemical bonds, or by ionic bonds, or by any combination thereof. Some hydrogels may be formed through the mixture of two or more materials that undergo chemical or physical reactions with each other to create the three-dimensional molecular network that provides the hydrogel with a degree of dimensional stability.

“Topical application” or “topical uses” means application to body surfaces, such as the skin, mucous membranes, vagina, oral cavity, internal surgical wound sites, and the like.

Terms “chromophore” and “photoactivator” are used herein interchangeably. A chromophore means a chemical compound, when contacted by light irradiation, is capable of absorbing the light. The chromophore readily undergoes photoexcitation and can transfer its energy to other molecules or emit it as light (fluorescence).

“Photobleaching” or “photobleaches” means the photochemical destruction of a chromophore. A chromophore may fully or partially photobleach.

The term “actinic light” is intended to mean light energy emitted from a specific light source (e.g. lamp, LED, or laser) and capable of being absorbed by matter (e.g. the chromophore or photoactivator). Terms “actinic light” and “light” are used herein interchangeably. In a preferred embodiment, the actinic light is visible light.

“Photopolymerization” herein refers to the use of visible or UV light to interact with light-sensitive compounds called “initiators” to create free radicals that can initiate polymerization of liquid or semi-liquid monomer or macromer to form a hydrogel.

“Skin rejuvenation” means a process of reducing, diminishing, retarding or reversing one or more signs of skin aging or generally improving the condition of skin. For instance, skin rejuvenation may include increasing luminosity of the skin, reducing pore size, reducing fine lines or wrinkles, improving thin and transparent skin, improving firmness, improving sagging skin (such as that produced by bone loss), improving dry skin (which might itch), reducing or reversing freckles, reducing or preventing the appearance of age spots, spider veins, rough and leathery skin, fine wrinkles that disappear when stretched, reducing loose skin, or improving a blotchy complexion. According to the present disclosure, one or more of the above conditions may be improved or one or more signs of aging may be reduced, diminished, retarded or even reversed by certain embodiments of the compositions, methods and uses of the present disclosure.

“Wound” means an injury to any tissue, including for example, acute, subacute, delayed or difficult to heal wounds, and chronic wounds. Examples of wounds may include both open and closed wounds. Wounds include, for example, amputations, burns, incisions, excisions, lesions, lacerations, abrasions, puncture or penetrating wounds, surgical wounds, amputations, contusions, hematomas, crushing injuries, ulcers (such as for example pressure, diabetic, venous or arterial), scarring (cosmesis), and wounds caused by periodontitis (inflammation of the periodontium).

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### (3) Biophotonic Hydrogels

The present disclosure provides, in a broad sense, biophotonic hydrogels and methods of using the biophotonic hydrogels. Biophotonic hydrogels can be, in a broad sense, activated by light (e.g., photons) of specific wavelength. Biophotonic hydrogel according to various embodiments of the present disclosure contains a polymerisable monomer, and at least one chromophore. The chromophore can absorb and/or emit light to initiate photopolymerization of the polymerisable monomer. In some embodiments, the chromophore is not fully photobleached after photopolymerization. Continued or repeated illumination of the biophotonic hydrogel can activate the at least one chromophore, which leads to light carrying on a therapeutic effect on its own, and/or to the photochemical activation of other agents contained in the composition.

When a chromophore absorbs a photon of a certain wavelength, it becomes excited. This is an unstable condition and the molecule tries to return to the ground state, giving away the excess energy. For some chromophores, it is favorable to emit the excess energy as light when returning to the ground state. This process is called fluorescence. The peak wavelength of the emitted fluorescence is shifted towards longer wavelengths compared to the absorption wavelengths due to loss of energy in the conversion process. This is called the Stokes' shift. In the proper environment (e.g., in a biophotonic hydrogel) much of this energy is transferred to the other components of the biophotonic hydrogel or to the treatment site directly.

Without being bound to theory, it is thought that fluorescent light emitted by photoactivated chromophores may have therapeutic properties due to its femto-, pico-, or nano-second emission properties which may be recognized by biological cells and tissues, leading to favourable biomodulation. Furthermore, the emitted fluorescent light has a longer wavelength and hence a deeper penetration into the tissue than the activating light. Irradiating tissue with such a broad range of wavelength, including in some embodiments the activating light which passes through the composition, may have different and complementary effects on the cells and tissues. In other words, chromophores are used in the biophotonic hydrogels of the present disclosure for therapeutic effect on tissues.

The biophotonic hydrogels of the present disclosure may have topical uses such as a mask or a wound dressing, or as an attachment to a light source, as a waveguide or as a light filter.

In addition the biophotonic materials can limit the contact between the chromophore and the tissue. These materials may be described based on the components making up the composition. Additionally or alternatively, the compositions of the present disclosure have functional and structural properties and these properties may also be used to define and describe the compositions. Individual components of the biophotonic hydrogels of the present disclosure, including chromophores, polymerisable monomers, cross linkers, initiators, catalysts, and other optional ingredients, such as thickening agents and surfactants, are detailed below.

The present disclosure also provides a premix composition to the material described herein, which will gel or polymerize upon light exposure. The premix composition comprises at least one chromophore and a polymerisable monomer, such as HEAA, which in its polymerized form is referred to as “PHEAA”.

(a) Chromophores

Suitable chromophores can be fluorescent compounds (or stains) (also known as “fluorochromes” or “fluorophores”). Other dye groups or dyes (biological and histological dyes, food colorings, carotenoids, and other dyes) can also be used. Suitable photoactivators can be those that are Generally Regarded As Safe (GRAS). Advantageously, photoactivators which are not well tolerated by the skin or other tissues can be included in the biophotonic hydrogel of the present disclosure, as in certain embodiments, the photoactivators are encapsulated within the hydrogel and may not contact the tissues.

In certain embodiments, the biophotonic hydrogel of the present disclosure comprises a first chromophore which undergoes partial or complete photobleaching upon application of light. In some embodiments, the first chromophore absorbs at a wavelength in the range of the visible spectrum, such as at a wavelength of about 380-800 nm, 380-700 nm, 400-800 nm, or 380-600 nm. In other embodiments, the first chromophore absorbs at a wavelength of about 200-800 nm, 200-700 nm, 200-600 nm or 200-500 nm. In some embodiments, the first chromophore absorbs at a wavelength of about 200-600 nm. In some embodiments, the first chromophore absorbs light at a wavelength of about 200-300 nm, 250-350 nm, 300-400 nm, 350-450 nm, 400-500 nm, 450-650 nm, 600-700 nm, 650-750 nm or 700-800 nm.

It will be appreciated to those skilled in the art that optical properties of a particular chromophore may vary depending on the chromophore's surrounding medium. Therefore, as used herein, a particular chromophore's absorption and/or emission wavelength (or spectrum) corresponds to the wavelengths (or spectrum) measured in a biophotonic hydrogel of the present disclosure.

The biophotonic hydrogel disclosed herein may include at least one additional chromophore. Combining chromophores may increase photo-absorption by the combined dye molecules and enhance absorption and photo-biomodulation selectivity. Thus, in certain embodiments, biophotonic hydrogels of the disclosure include more than one chromophore. When such multi-chromophore materials are illuminated with light, energy transfer can occur between the chromophores. This process, known as resonance energy transfer, is a widely prevalent photophysical process through which an excited ‘donor’ chromophore (also referred to herein as first chromophore) transfers its excitation energy to an ‘acceptor’ chromophore (also referred to herein as second chromophore). The efficiency and directedness of resonance energy transfer depends on the spectral features of donor and acceptor chromophores. In particular, the flow of energy between chromophores is dependent on a spectral overlap reflecting the relative positioning and shapes of the absorption and emission spectra. More specifically, for energy transfer to occur, the emission spectrum of the donor chromophore must overlap with the absorption spectrum of the acceptor chromophore.

Energy transfer manifests itself through decrease or quenching of the donor emission and a reduction of excited state lifetime accompanied also by an increase in acceptor emission intensity. To enhance the energy transfer efficiency, the donor chromophore should have good abilities to absorb photons and emit photons. Furthermore, the more overlap there is between the donor chromophore's emission spectra and the acceptor chromophore's absorption spectra, the better a donor chromophore can transfer energy to the acceptor chromophore.

In certain embodiments, where the biophotonic hydrogels of the present disclosure further comprise a second chromophore, the first chromophore may have an emission spectrum that overlaps at least about 80%, about 75%, about 70%, about 65%, about 60%, about 55%, about 50%, about 45%, about 40%, about 35%, about 30%, about 25%, about 20%, about 15% or about 10% with an absorption spectrum of the second chromophore. In some embodiments, the first chromophore has an emission spectrum that overlaps at least about 20% with an absorption spectrum of the second chromophore. In some embodiments, the first chromophore has an emission spectrum that overlaps at least between about 1-10%, between about 5-15%, between about 10-20%, between about 15-25%, between about 20-30%, between about 25-35%, between about 30-40%, between about 35-45%, between about 50-60%, between about 55-65% or between about 60-70% with an absorption spectrum of the second chromophore.

% spectral overlap, as used herein, means the % overlap of a donor chromophore's emission wavelength range with an acceptor chromophore's absorption wavelength rage, measured at spectral full width quarter maximum (FWQM). In some embodiments, the second chromophore absorbs at a wavelength in the range of the visible spectrum. In certain embodiments, the second chromophore has an absorption wavelength that is relatively longer than that of the first chromophore within the range of about 50-250, 25-150 or 10-100 nm.

The chromophore can be present in an amount of about 0.001-40% per weight of the biophotonic hydrogel. In certain embodiments, the first chromophore is present in an amount of between about 0.001-3%, between about 0.001-0.01%, between about 0.005-0.1%, between about 0.1-0.5%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the biophotonic hydrogel.

In embodiments comprising a second chromophore, the second chromophore can be present in an amount of about 0.001-40% per weight of the biophotonic hydrogel. In some embodiments, the second chromophore is present in an amount of between about 0.001-3%, between about 0.001-0.01%, between about 0.005-0.1%, between about 0.1-0.5%, between about 0.5-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40% per weight of the biophotonic hydrogel.

In certain embodiments, the total weight per weight of chromophore or combination of chromophores may be in the amount of between about 0.005-1%, between about 0.05-2%, between about 1-5%, between about 2.5-7.5%, between about 5-10%, between about 7.5-12.5%, between about 10-15%, between about 12.5-17.5%, between about 15-20%, between about 17.5-22.5%, between about 20-25%, between about 22.5-27.5%, between about 25-30%, between about 27.5-32.5%, between about 30-35%, between about 32.5-37.5%, or between about 35-40.001% per weight of the biophotonic hydrogel.

The concentration of the chromophore to be used can be selected based on the desired intensity and duration of the biophotonic activity from the biophotonic hydrogel, and on the desired medical or cosmetic effect. For example, some dyes such as xanthene dyes reach a ‘saturation concentration’ after which further increases in concentration do not provide substantially higher emitted fluorescence. Further increasing the chromophore concentration above the saturation concentration can reduce the amount of activating light passing through the matrix. Therefore, if more fluorescence is required for a certain application than activating light, a high concentration of chromophore can be used. However, if a balance is required between the emitted fluorescence and the activating light, a concentration close to or lower than the saturation concentration can be chosen.

Suitable chromophores that may be used in the biophotonic hydrogels of the present disclosure include, but are not limited to the following:

Chlorophyll Dyes

Exemplary chlorophyll dyes include but are not limited to chlorophyll a; chlorophyll b; chlorophyllin; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

Xanthene Derivatives

Exemplary xanthene dyes include but are not limited to eosin, eosin B (4′,5′-dibromo,2′,7′-dinitr-o-fluorescein, dianion); eosin Y; eosin Y (2′,4′,5′,7′-tetrabromo-fluorescein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines that include, but are not limited to, 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

Methylene Blue Dyes

Exemplary methylene blue derivatives include, but are not limited to, 1-methyl methylene blue; 1,9-dimethyl methylene blue; methylene blue; methylene blue (16 μM); methylene blue (14 μM); methylene violet; bromomethylene violet; 4-iodomethylene violet; 1,9-dimethyl-3-dimethyl-amino-7-diethyl-a-mino-phenothiazine; and 1,9-dimethyl-3-diethylamino-7-dibutyl-amino-phenot-hiazine.

Azo Dyes

Exemplary azo (or diazo-) dyes include but are not limited to methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

In some aspects of the disclosure, the one or more chromophores of the biophotonic hydrogels disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin Green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural green 3(chlorophyllin), Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, Picric acid, Ponceau 2R, Ponceau 6R, Ponceau B, Ponceau de Xylidine, Ponceau S, Primula, Purpurin, Pyronin B, phycobilins, Phycocyanins, Phycoerythrins. Phycoerythrincyanin (PEC), Phthalocyanines, Pyronin G, Pyronin Y, Quinine, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Vitamin B, Water blue I, Water soluble eosin, Xylidine ponceau, or Yellowish eosin.

In certain embodiments, the biophotonic hydrogel of the present disclosure includes any of the chromophores listed above, or a combination thereof, so as to provide a synergistic biophotonic effect at the application site.

Without being bound to any particular theory, a synergistic effect of the chromophore combinations means that the biophotonic effect is greater than the sum of their individual effects. Advantageously, this may translate to increased reactivity of the biophotonic hydrogel, faster or improved treatment time. Also, the treatment conditions need not be altered to achieve the same or better treatment results, such as time of exposure to light, power of light source used, and wavelength of light used. In other words, use of synergistic combinations of chromophores may allow the same or better treatment without necessitating a longer time of exposure to a light source, a higher power light source or a light source with different wavelengths.

In some embodiments, the biophotonic hydrogel includes Eosin Y as a first chromophore and any one or more of Rose Bengal, Fluorescein, Erythrosine, Phloxine B, chlorophyllin as a second chromophore. It is believed that these combinations have a synergistic effect as they can transfer energy to one another when activated due in part to overlaps or close proximity of their absorption and emission spectra. This transferred energy is then emitted as fluorescence and/or leads to production of reactive oxygen species. This absorbed and re-emitted light is thought to be transmitted throughout the composition, and also to be transmitted into the site of treatment.

In further embodiments, the material includes the following synergistic combinations: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine. Other synergistic chromophore combinations are also possible.

By means of synergistic effects of the chromophore combinations in the biophotonic hydrogel, chromophores which cannot normally be activated by an activating light (such as a blue light from an LED), can be activated through energy transfer from chromophores which are activated by the activating light. In this way, the different properties of photoactivated chromophores can be harnessed and tailored according to the cosmetic or the medical therapy required.

For example, Rose Bengal can generate a high yield of singlet oxygen when activated in the presence of molecular oxygen, however it has a low quantum yield in terms of emitted fluorescent light. Rose Bengal has a peak absorption around 540 nm and so can be activated by green light. Eosin Y has a high quantum yield and can be activated by blue light. By combining Rose Bengal with Eosin Y, one obtains a composition which can emit therapeutic fluorescent light and generate singlet oxygen when activated by blue light. In this case, the blue light photoactivates Eosin Y which transfers some of its energy to Rose Bengal as well as emitting some energy as fluorescence.

In some embodiments, the chromophore or chromophores are selected such that their emitted fluorescent light, on photoactivation, is within one or more of the green, yellow, orange, red and infrared portions of the electromagnetic spectrum, for example having a peak wavelength within the range of about 490 nm to about 800 nm. In certain embodiments, the emitted fluorescent light has a power density of between 0.005 to about 10 mW/cm², about 0.5 to about 5 mW/cm².

(b) Polymerisable Monomers

The polymerisable monomers can be a hydrophilic monomer. As used herein, a hydrophilic monomer refers to any monomer which, when polymerized, yields a hydrophilic polymer capable of forming a hydrogel when contacted with an aqueous medium such as water. In some embodiments, a hydrophilic monomer can contain a functional group in the polymer backbone or as lateral chains. The term “functional group” as used herein refers to a chemical moiety which exhibits bond formation capability. Examples of functional group include, but are not limited to, hydroxyl (—OH), carboxyl (—COOH), amide (—CONH—), thiol (—SH), or sulfonic (—SO3H) groups. Examples of hydrophilic monomers include, but are not limited to, hydroxyl-containing monomers such as 2-hydroxyethyl methacrylate, 2-hydroxyethyl acrylate, 2-hydroxyethyl methacrylamide, 2-hydroxyethyl acrylamide, N-2-hydroxyethyl vinyl carbamate, 2-hydroxyethyl vinyl carbonate, 2-hydroxypropyl methacrylate, hydroxyhexyl methacrylate and hydroxyoctyl methacrylate; carboxyl-containing monomers such as acrylic acid, methacrylic acid, itaconic acid, fumaric acid, crotonic acid, maleic acid and salts thereof, esters containing free carboxyl groups of unsaturated polycarboxylic acids, such as monomethyl maleate ester, monoethyl maleate ester, monomethyl fumarate ester, monoethyl fumarate ester and salts thereof; amide-containing monomers such as (meth)acrylamide, crotonic amide, cinnamic amide, maleic diamide and fumaric diamide; thiol-containing monomers such as methanethiole, ethanethiol, 1-propanethiol, butanethiol, tert-butyl mercaptan, and pentanethiols; sulfonic acid-containing monomers such as p-styrenesulfonic acid, vinylsulfonic acid, p-a-methylstyrenesulfonic acid, isoprene sulfonide and salts thereof.

In certain aspects of the present disclosure the polymerisable monomer is N-Hydroxyethyl acrylamide (HEAA). In certain embodiments of the disclosure the HEAA is present in the biophotonic hydrogel composition in the amount of about 1-50 wt %, or about 5-50 wt %, or about 5-40 wt %, or about 10-30 wt %, or about 15-25 wt % or about 20 wt % HEAA.

(c) Cross Linkers

The cross-linking agent of the present disclosure is intended to form a cross-linked structure during the process of polymerization. Typical examples of cross-linking agents include, but are not limited to, compounds having at least two polymerizable unsaturated double bonds in the molecular unit thereof, compounds having at least two groups capable of reacting with a functional group such as acid group, hydroxyl groups, amino group. in the molecule; compounds having at least one double bond and at least one group capable of reacting with the functional group of the monomer compounds having at least two points capable of reacting with the functional group of monomer within the molecular unit; and hydrophilic polymers capable of forming a cross-linked structure as by graft bondage during the process of polymerization of the monomer component may be cited.

Some embodiments of the biophotonic hydrogels of the present disclosure have a cross-linking agent comprised of: poly(ethylene glycol) diacrylate, or polyvalent(meth)acrylamide compounds such as N,N′-methylene bis(meth)acrylamide; or poly(meth)acrylate compounds such as poly(ethylene glycol) di(meth)acrylate, poly(propylene) glycol di(meth)acrylate, glycerol di(meth)acrylate, glycerol acrylate methacrylate, trimethylolpropane di (meth) acrylate, trimethylol propane acrylate methacrylate, pentaerythritol di(meth)acrylate, glycerol tri (meth) acrylate, trimethylolpropane tri (meth) acrylate, pentaerythritol tri(meth)acrylate, and pentaerythritol tetra-(meth)acrylate; or polyallyl compounds such as triallyl amine, poly(allyloxy) alkane, triallyl cyanurate, triallyl isocyanurate, and triallyl phosphate; or polyglycidyl compounds such as poly(ethylene glycol) diglycidyl ether, propylene glycol diglycidyl ether, glycerol diglycidyl ether, and glycerol triglycidyl ether; polyisocyanate compounds such as 2,4-toluylene diisocyanate and hexamethylene diisocyanate; polyoxazoline compounds; or reactive group-containing (meth)acryl amides or (meth)acrylates such as N-methylol (meth)acryl amide and glycidyl (meth)acrylate.

It is well known to persons of ordinary skill in the art that a decrease in the density of cross-links adds to the absorption capacity and, at the same time, increases the content of soluble component. The amount of cross-linking agent employed in the current disclosure can be varied. In certain embodiments of the present disclosure the cross-linking agent is poly(ethylene glycol) diacrylate (PEGDA). In further embodiments of the present disclosure the PEGDA is present in the biophotonic hydrogel composition in the amount of 0.1-10 wt %, or 1-5 wt % of the total composition.

(d) Initiators

Certain embodiments of biophotonic hydrogel of the present disclosure may also comprise a polymerization initiator. As used herein, an “initiator” for a polymerization reaction refers to a compound that can start a polymerization reaction, typically by providing a free radical species. The free radical species can be generated directly by the initiator compound, or can be abstracted from a compound that facilitates initiation of polymerization. An initiator molecule of the present disclosure may be a photoinitator, meaning it can be activated by light. The free radicals generated or abstracted by the activated initiator compound can then propagate radical chain polymerization. Initiator molecules of the present disclosure may include triethanolamine (TEA). In some embodiments of the biophotonic hydrogel material may comprise between about 0-1 wt %, between about 0.1-0.5 wt %, between about 0.2-1.0 wt %, between about 0.25-1.25 wt %, between about 0.1-2.0 wt %, between about 0.2-4.0 wt % TEA.

(e) Catalysts

Certain embodiments of biophotonic hydrogel of the present disclosure may also comprise a catalyst. As used herein, a “catalyst” for a polymerization reaction refers to a compound that can assist the polymerization of polymerizable material following initiation of the reaction. Generally, a catalyst will promote completion of the polymerization reaction and/or increase the rate that the polymerizable material becomes incorporated into a polymerized product. Catalysts of the disclosure may be incorporated into the polymerized product and provide the product with (an) improved biocompatible feature(s). Suitable accelerators are generally lower molecular weight monomeric-type compounds that enhance matrix formation when added to and polymerized with a macromer-containing composition. A catalyst of the present disclosure may include 1-vinyl-2 pyrrolidinone (NVP). In certain embodiments the catalyst is NVP. In some embodiments of the biophotonic hydrogel material may comprise between about 0-1 wt %, between about 0.1-0.5 wt %, between about 0.2-1.0 wt %, between about 0.25-1.25 wt %, between about 0.1-2.0 wt %, between about 0.2-4.0 wt % NVP.

(f) Surfactants

The biophotonic hydrogel of the present disclosure may also comprise a surfactant. The surfactant may be present in an amount of about 5-10%, or about 10-15%, or about 15-20%, or about 20-25%, or about 25-30% of the total composition by weight. In certain embodiments the surfactant is a Poloxamer. Poloxamers are commercially available from BASF Corporation. Poloxamers produce reverse thermal gelatin compositions, i.e., with the characteristic that their viscosity increases with increasing temperature up to a point from which viscosity again decreases. In certain embodiments of the disclosure, the surfactant is Pluronic® F127 (also known as Poloxamer 407). In some embodiments, the biophotonic hydrogel material may comprise Pluronic® F127 in the amount of 1-25 wt % of the total composition. In some embodiments, the biophotonic hydrogel material may comprise between about 1-5 wt %, between about 2.5-7.5 wt %, between about 5-10 wt %, between about 7.5-12.5 wt %, between about 10-15 wt %, between about 12.5-17.5 wt %, between about 15-20 wt %, between about 20-25 wt % Pluronic® F127. In certain embodiments, the biophotonic hydrogel comprises a further surfactant comprising a cationic surfactant. In certain other embodiments, the cationic surfactant is cetyltrimethyl ammonium bromide (CTAB). In certain other embodiments, the cationic surfactant is cetyltrimethyl ammonium bromide (CTAB). In certain embodiments, the CTAB may be present in the biophotonic hydrogel at a percentage concentration to allow for a formation of micelles by the CTAB (termed a critical micelle concentration). In certain embodiments, the critical micelle concentration may be increased with an increase in incubation temperature of the biophotonic hydrogel.

(g) Thickening Agents

In certain embodiments, the biophotonic hydrogel may also include thickening agents or stabilizers such as gelatin and/or modified celluloses such as hydroxyethyl cellulose (HEC) and carboxymethyl cellulose (CMC), and/or polysaccharides such as xanthan gum, guar gum, and/or starches and/or any other thickening agent. In certain embodiments of the disclosure, the stabilizer or thickening agent may comprise gelatin. For example, the biophotonic hydrogel may comprise about 0-5 wt %, about 1-5 wt %, about 1.5-10 wt %, or about 2-20 wt % gelatin. In other embodiments of the disclosure, the stabilizer or thickening agent may comprise HEC. For example, the biophotonic hydrogel may comprise between about 0-2.5 wt %, between about 1-5 wt %, between about 1.5-10 wt % HEC.

(h) Mechanical Strengtheners

In some embodiments, the biophotonic hydrogel composition further comprises an additional compound that may enhance the mechanical strength of the biophotonic hydrogel. In some embodiments, the additional compound may be a silica-based compound. In certain embodiments, the silica-based compound may be a silica clay or fumed silica (SiO₂). In certain embodiments, the silica clay may be bentonite (B). The bentonite surfactant may be present in the biophotonic hydrogel at between about 0.01-0.5 wt %, between about 0.25-0.75 wt %, between about 0.5-0.75 wt %, between about 0.75-1.0 wt % of the biophotonic hydrogel. The fumed silica surfactant may be present in the biophotonic hydrogel at between about 0.01-1.0 wt %, between about 1.0-2.0 wt %, between about 2.0-3.0 wt %, between about 3.0-4.0 wt %, between about 4.0-5.0 wt % of the biophotonic hydrogel.

In certain other embodiments, the biophotonic hydrogel comprises a combination of the further surfactant and the additional compound for enhancing the mechanical strength of the biophotonic hydrogel. In certain other embodiments, the combination of the further surfactant and the additional compound for enhancing the mechanical strength in the biophotonic hydrogel comprises CTAB and fumed silica.

(i) Antimicrobials

Antimicrobials kill microbes or inhibit their growth or accumulation, and are optionally included in the biophotonic hydrogels of the present disclosure. Exemplary antimicrobials (or antimicrobial agent) are recited in U.S. Patent Application Publication Nos: 2004/0009227 and 2011/0081530. Suitable antimicrobials for use in the methods and compositions of the present disclosure include, but not limited to, hydrogen peroxide, urea hydrogen peroxide, benzoyl peroxide, phenolic and chlorinated phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

Hydrogen peroxide (H₂O₂) is a powerful oxidizing agent, and breaks down into water and oxygen and does not form any persistent, toxic residual compound. A suitable range of concentration over which hydrogen peroxide can be used in the biophotonic hydrogel is from about 0.1% to about 3%, about 0.1 to 1.5%, about 0.1% to about 1%, about 1%, less than about 1%.

Urea hydrogen peroxide (also known as urea peroxide, carbamide peroxide or percarbamide) is soluble in water and contains approximately 35% hydrogen peroxide. A suitable range of concentration over which urea peroxide can be used in the biophotonic hydrogel of the present disclosure is less than about 0.25%, or less than about 0.3%, from 0.001 to 0.25%, or from about 0.3% to about 5%. Urea peroxide breaks down to urea and hydrogen peroxide in a slow-release fashion that can be accelerated with heat or photochemical reactions.

Benzoyl peroxide consists of two benzoyl groups (benzoic acid with the H of the carboxylic acid removed) joined by a peroxide group. It is found in treatments for acne, in concentrations varying from 2.5% to 10%. The released peroxide groups are effective at killing bacteria. Benzoyl peroxide also promotes skin turnover and clearing of pores, which further contributes to decreasing bacterial counts and reduce acne. Benzoyl peroxide breaks down to benzoic acid and oxygen upon contact with skin, neither of which is toxic. A suitable range of concentration over which benzoyl peroxide can be used in the biophotonic hydrogel is from about 2.5% to about 5%.

According to certain embodiments, the biophotonic hydrogel of the present disclosure may optionally comprise one or more additional components, such as oxygen-rich compounds as a source of oxygen radicals. Peroxide compounds are oxidants that contain the peroxy group (R—O—O—R), which is a chainlike structure containing two oxygen atoms, each of which is bonded to the other and a radical or some element. When a biophotonic material of the present disclosure comprising an oxidant is illuminated with light, the chromophores are excited to a higher energy state. When the chromophores' electrons return to a lower energy state, they emit photons with a lower energy level, thus causing the emission of light of a longer wavelength (Stokes' shift). In the proper environment, some of this energy is transferred to oxygen or the reactive hydrogen peroxide and causes the formation of oxygen radicals, such as singlet oxygen. The singlet oxygen and other reactive oxygen species generated by the activation of the biophotonic material are thought to operate in a hormetic fashion. That is, a health beneficial effect that is brought about by the low exposure to a normally toxic stimuli (e.g. reactive oxygen), by stimulating and modulating stress response pathways in cells of the targeted tissues. Endogenous response to exogenous generated free radicals (reactive oxygen species) is modulated in increased defense capacity against the exogenous free radicals and induces acceleration of healing and regenerative processes. Furthermore, activation of the oxidant may also produce an antibacterial effect. The extreme sensitivity of bacteria to exposure to free radicals makes the biophotonic hydrogel of the present disclosure potentially a bactericidal composition.

Specific phenolic and chlorinated phenolic antimicrobial agents that can be used in the disclosure include, but are not limited to: phenol; 2-methyl phenol; 3-methyl phenol; 4-methyl phenol; 4-ethyl phenol; 2,4-dimethyl phenol; 2,5-dimethyl phenol; 3,4-dimethyl phenol; 2,6-dimethyl phenol; 4-n-propyl phenol; 4-n-butyl phenol; 4-n-amyl phenol; 4-tert-amyl phenol; 4-n-hexyl phenol; 4-n-heptyl phenol; mono- and poly-alkyl and aromatic halophenols; p-chlorophenyl; methyl p-chlorophenol; ethyl p-chlorophenol; n-propyl p-chlorophenol; n-butyl p-chlorophenol; n-amyl p-chlorophenol; sec-amyl p-chlorophenol; n-hexyl p-chlorophenol; cyclohexyl p-chlorophenol; n-heptyl p-chlorophenol; n-octyl; p-chlorophenol; o-chlorophenol; methyl o-chlorophenol; ethyl o-chlorophenol; n-propyl o-chlorophenol; n-butyl o-chlorophenol; n-amyl o-chlorophenol; tert-amyl o-chlorophenol; n-hexyl o-chlorophenol; n-heptyl o-chlorophenol; o-benzyl p-chlorophenol; o-benxyl-m-methyl p-chlorophenol; o-benzyl-m,m-dimethyl p-chlorophenol; o-phenylethyl p-chlorophenol; o-phenylethyl-m-methyl p-chlorophenol; 3-methyl p-chlorophenol 3,5-dimethyl p-chlorophenol, 6-ethyl-3-methyl p-chlorophenol, 6-n-propyl-3-methyl p-chlorophenol; 6-iso-propyl-3-methyl p-chlorophenol; 2-ethyl-3,5-dimethyl p-chlorophenol; 6-sec-butyl-3-methyl p-chlorophenol; 2-iso-propyl-3,5-dimethyl p-chlorophenol; 6-diethylmethyl-3-methyl p-chlorophenol; 6-iso-propyl-2-ethyl-3-methyl p-chlorophenol; 2-sec-amyl-3,5-dimethyl p-chlorophenol; 2-diethylmethyl-3,5-dimethyl p-chlorophenol; 6-sec-octyl-3-methyl p-chlorophenol; p-chloro-m-cresol p-bromophenol; methyl p-bromophenol; ethyl p-bromophenol; n-propyl p-bromophenol; n-butyl p-bromophenol; n-amyl p-bromophenol; sec-amyl p-bromophenol; n-hexyl p-bromophenol; cyclohexyl p-bromophenol; o-bromophenol; tert-amyl o-bromophenol; n-hexyl o-bromophenol; n-propyl-m,m-dimethyl o-bromophenol; 2-phenyl phenol; 4-chloro-2-methyl phenol; 4-chloro-3-methyl phenol; 4-chloro-3,5-dimethyl phenol; 2,4-dichloro-3,5-dimethylphenol; 3,4,5,6-tetabromo-2-methylphenol-; 5-methyl-2-pentylphenol; 4-isopropyl-3-methylphenol; para-chloro-metaxylenol (PCMX); chlorothymol; phenoxyethanol; phenoxyisopropanol; and 5-chloro-2-hydroxydiphenylmethane.

Resorcinol and its derivatives can also be used as antimicrobial agents. Specific resorcinol derivatives include, but are not limited to: methyl resorcinol; ethyl resorcinol; n-propyl resorcinol; n-butyl resorcinol; n-amyl resorcinol; n-hexyl resorcinol; n-heptyl resorcinol; n-octyl resorcinol; n-nonyl resorcinol; phenyl resorcinol; benzyl resorcinol; phenylethyl resorcinol; phenylpropyl resorcinol; p-chlorobenzyl resorcinol; 5-chloro-2,4-dihydroxydiphenyl methane; 4′-chloro-2,4-dihydroxydiphenyl methane; 5-bromo-2,4-dihydroxydiphenyl methane; and 4′-bromo-2,4-dihydroxydiphenyl methane.

Specific bisphenolic antimicrobial agents that can be used in the disclosure include, but are not limited to: 2,2′-methylene bis-(4-chlorophenol); 2,4,4′trichloro-2′-hydroxy-diphenyl ether, which is sold by Ciba Geigy, Florham Park, N.J. under the tradename Triclosan®; 2,2′-methylene bis-(3,4,6-trichlorophenol); 2,2′-methylene bis-(4-chloro-6-bromophenol); bis-(2-hydroxy-3,5-dichlorop-henyl) sulphide; and bis-(2-hydroxy-5-chlorobenzyl)sulphide.

Specific benzoie esters (parabens) that can be used in the disclosure include, but are not limited to: methylparaben; propylparaben; butylparaben; ethylparaben; isopropylparaben; isobutylparaben; benzylparaben; sodium methylparaben; and sodium propylparaben.

Specific halogenated carbanilides that can be used in the disclosure include, but are not limited to: 3,4,4′-trichlorocarbanilides, such as 3-(4-chlorophenyl)-1-(3,4-dichlorphenyl)urea sold under the tradename Triclocarban® by Ciba-Geigy, Florham Park, N.J.; 3-trifluoromethyl-4,4′-dichlorocarbanilide; and 3,3′,4-trichlorocarbanilide.

Specific polymeric antimicrobial agents that can be used in the disclosure include, but are not limited to: polyhexamethylene biguanide hydrochloride; and poly(iminoimidocarbonyl iminoimidocarbonyl iminohexamethylene hydrochloride), which is sold under the tradename Vantocil® IB.

Specific thazolines that can be used in the disclosure include, but are not limited to that sold under the tradename Micro-Check®; and 2-n-octyl-4-isothiazolin-3-one, which is sold under the tradename Vinyzene® IT-3000 DIDP.

Specific trichloromethylthioimides that can be used in the disclosure include, but are not limited to: N-(trichloromethylthio)phthalimide, which is sold under the tradename Fungitrol®; and N-trichloromethylthio-4-cyclohexene-1,2-dicarboximide, which is sold under the tradename Vancide®.

Specific natural antimicrobial agents that can be used in the disclosure include, but are not limited to, oils of: anise; lemon; orange; rosemary; wintergreen; thyme; lavender; cloves; hops; tea tree; citronella; wheat; barley; lemongrass; cedar leaf; cedarwood; cinnamon; fleagrass; geranium; sandalwood; violet; cranberry; eucalyptus; vervain; peppermint; gum benzoin; basil; fennel; fir; balsam; menthol; ocmea origanuin; hydastis; carradensis; Berberidaceac daceae; Ratanhiae longa; and Curcuma longa. Also included in this class of natural antimicrobial agents are the key chemical components of the plant oils which have been found to provide antimicrobial benefit. These chemicals include, but are not limited to: anethol; catechole; camphene; thymol; eugenol; eucalyptol; ferulic acid; farnesol; hinokitiol; tropolone; limonene; menthol; methyl salicylate; carvacol; terpineol; verbenone; berberine; ratanhiae extract; caryophellene oxide; citronellic acid; curcumin; nerolidol; and geraniol.

Specific metal salts that can be used in the disclosure include, but are not limited to, salts of metals in groups 3a-5a, 3b-7b, and 8 of the periodic table. Specific examples of metal salts include, but are not limited to, salts of: aluminum; zirconium; zinc; silver; gold; copper; lanthanum; tin; mercury; bismuth; selenium; strontium; scandium; yttrium; cerium; praseodymiun; neodymium; promethum; samarium; europium; gadolinium; terbium; dysprosium; holmium; erbium; thalium; ytterbium; lutetium; and mixtures thereof. An example of the metal-ion based antimicrobial agent is sold under the tradename HealthShield®, and is manufactured by HealthShield Technology, Wakefield, Mass.

Specific broad-spectrum antimicrobial agents that can be used in the disclosure include, but are not limited to, those that are recited in other categories of antimicrobial agents herein.

Additional antimicrobial agents that can be used in the methods of the disclosure include, but are not limited to: pyrithiones, and in particular pyrithione-including zinc complexes such as that sold under the tradename Octopirox®; dimethyidimethylol hydantoin, which is sold under the tradename Glydant®; methylchloroisothiazolinone/methylisothiazolinone, which is sold under the tradename Kathon CG®; sodium sulfite; sodium bisulfite; imidazolidinyl urea, which is sold under the tradename Germall 115®; diazolidinyl urea, which is sold under the tradename Germall 118; benzyl alcohol v2-bromo-2-nitropropane-1,3-diol, which is sold under the tradename Bronopol®; formalin or formaldehyde; iodopropenyl butylcarbamate, which is sold under the tradename Polyphase P100®; chloroacetamide; methanamine; methyldibromonitrile glutaronitrile (1,2-dibromo-2,4-dicyanobutane), which is sold under the tradename Tektamer®; glutaraldehyde; 5-bromo-5-nitro-1,3-dioxane, which is sold under the tradename Bronidox®; phenethyl alcohol; o-phenylphenol/sodium o-phenylphenol sodium hydroxymethylglycinate, which is sold under the tradename Suttocide A®; polymethoxy bicyclic oxazolidine; which is sold under the tradename Nuosept C®; dimethoxane; thimersal; dichlorobenzyl alcohol; captan; chlorphenenesin; dichlorophen; chlorbutanol; glyceryl laurate; halogenated diphenyl ethers; 2,4,4′-trichloro-2′-hydroxy-diphenyl ether, which is sold under the tradename Triclosan® and is available from Ciba-Geigy, Florham Park, N.J.; and 2,2′-dihydroxy-5,5′-dibromo-diphenyl ether.

Additional antimicrobial agents that can be used in the methods of the disclosure include those disclosed by U.S. Pat. Nos. 3,141,321; 4,402,959; 4,430,381; 4,533,435; 4,625,026; 4,736,467; 4,855,139; 5,069,907; 5,091,102; 5,639,464; 5,853,883; 5,854,147; 5,894,042; and 5,919,554, and U.S. Pat. Appl. Publ. Nos. 20040009227 and 20110081530.

### (4) Optical Properties of the Biophotonic Materials

In certain embodiments, the biophotonic hydrogels of the present disclosure are substantially transparent or translucent. The % transmittance of the biophotonic hydrogel can be measured in the range of wavelengths from 250 nm to 800 nm using, for example, a Perkin-Elmer Lambda 9500 series UV-visible spectrophotometer. In some embodiments, transmittance within the visible range is measured and averaged. In some other embodiments, transmittance of the biophotonic hydrogel is measured with the chromophore omitted. As transmittance is dependent upon thickness, the thickness of each sample can be measured with calipers prior to loading in the spectrophotometer. Transmittance values can be normalized according to

$${F}_{T - corr} ( λ , {t}_{2} ) = {[ {e}^{- {σ}_{t}} ( λ ) {t}_{1} ]}^{\frac{{t}_{2}}{{t}_{1}}} = {[ {F}_{T - corr} ( λ , {t}_{1} ) ]}^{\frac{{t}_{2}}{{t}_{1}}} ,$$

where t₁=actual specimen thickness, t₂=thickness to which transmittance measurements can be normalized. In the art, transmittance measurements are usually normalized to 1 cm.

In some embodiments, the biophotonic hydrogel has a transmittance that is more than about 20%, 30%, 40%, 50%, 60%, 70%, or 75% within the visible range. In some embodiments, the transmittance exceeds 40%, 41%, 42%, 43%, 44%, or 45% within the visible range.

### (5) Methods of Use

The biophotonic hydrogels of the present disclosure may have cosmetic and/or medical benefits. They can be used to promote skin rejuvenation and skin conditioning, promote the treatment of a skin disorder such as acne, eczema, dermatitis or psoriasis, promote tissue repair, and modulate inflammation, modulate collagen synthesis, reduce or avoid scarring, for cosmesis, or promote wound healing including reducing the depth of periodontitis pockets. They can be used to treat acute inflammation. Acute inflammation can present itself as pain, heat, redness, swelling and loss of function, and includes inflammatory responses such as those seen in allergic reactions such as those to insect bites e.g.; mosquito, bees, wasps, poison ivy, or post-ablative treatment.

Accordingly, in certain embodiments, the present disclosure provides a method for treating acute inflammation.

In certain embodiments, the present disclosure provides a method for providing skin rejuvenation or for improving skin condition, treating a skin disorder, preventing or treating scarring, and/or accelerating wound healing and/or tissue repair, the method comprising: applying a biophotonic hydrogel of the present disclosure to the area of the skin or tissue in need of treatment, and illuminating the biophotonic hydrogel premix with light having a wavelength that overlaps with an absorption spectrum of the chromophore(s) present in the biophotonic hydrogel to induce the formation of the hydrogel; and continued or repeated illumination of the biophotonic hydrogel with light having a wavelength that overlaps with an absorption spectrum of the chromophore(s) present in the biophotonic hydrogel.

In the methods of the present disclosure, any source of actinic light can be used. Any type of halogen, LED or plasma arc lamp, or laser may be suitable. The primary characteristic of suitable sources of actinic light will be that they emit light in a wavelength (or wavelengths) appropriate for activating the one or more photoactivators present in the composition. In one embodiment, an argon laser is used. In another embodiment, a potassium-titanyl phosphate (KTP) laser (e.g. a GreenLight™ laser) is used. In yet another embodiment, a LED lamp such as a photocuring device is the source of the actinic light. In yet another embodiment, the source of the actinic light is a source of light having a wavelength between about 200 to 800 nm. In another embodiment, the source of the actinic light is a source of visible light having a wavelength between about 400 and 600 nm. In another embodiment, the source of the actinic light is a source of visible light having a wavelength between about 400 and 700 nm. In yet another embodiment, the source of the actinic light is blue light. In yet another embodiment, the source of the actinic light is red light. In yet another embodiment, the source of the actinic light is green light. Furthermore, the source of actinic light should have a suitable power density. Suitable power density for non-collimated light sources (LED, halogen or plasma lamps) are in the range from about 0.1 mW/cm² to about 200 mW/cm². Suitable power density for laser light sources are in the range from about 0.5 mW/cm² to about 0.8 mW/cm².

In some embodiments of the methods of the present disclosure, the light has an energy at the subject's skin surface of between about 0.1 mW/cm² and about 500 mW/cm², or 0.1-300 mW/cm², or 0.1-200 mW/cm², wherein the energy applied depends at least on the condition being treated, the wavelength of the light, the distance of the skin from the light source and the thickness of the biophotonic material. In certain embodiments, the light at the subject's skin is between about 1-40 mW/cm², or between about 20-60 mW/cm², or between about 40-80 mW/cm², or between about 60-100 mW/cm², or between about 80-120 mW/cm², or between about 100-140 mW/cm², or between about 30-180 mW/cm², or between about 120-160 mW/cm², or between about 140-180 mW/cm², or between about 160-200 mW/cm², or between about 110-240 mW/cm², or between about 110-150 mW/cm², or between about 190-240 mW/cm².

The activation of the chromophore(s) within the biophotonic hydrogel may take place almost immediately on illumination (femto- or pico seconds). A prolonged exposure period may be beneficial to exploit the synergistic effects of the absorbed, reflected and reemitted light of the biophotonic hydrogel of the present disclosure and its interaction with the tissue being treated. In one embodiment, the time of exposure of the tissue or skin or the biophotonic hydrogel to actinic light is a period between 0.01 minutes and 90 minutes. In another embodiment, the time of exposure of the tissue or skin or the biophotonic hydrogel to actinic light is a period between 1 minute and 5 minutes. In some other embodiments, the biophotonic hydrogel is illuminated for a period between 1 minute and 3 minutes. In certain embodiments, light is applied for a period of about 1-30 seconds, about 15-45 seconds, about 30-60 seconds, about 0.75-1.5 minutes, about 1-2 minutes, about 1.5-2.5 minutes, about 2-3 minutes, about 2.5-3.5 minutes, about 3-4 minutes, about 3.5-4.5 minutes, about 4-5 minutes, about 5-10 minutes, about 10-15 minutes, about 15-20 minutes, or about 20-30 minutes. The treatment time may range up to about 90 minutes, about 80 minutes, about 70 minutes, about 60 minutes, about 50 minutes, about 40 minutes or about 30 minutes. It will be appreciated that the treatment time can be adjusted in order to maintain a dosage by adjusting the rate of fluence delivered to a treatment area. For example, the delivered fluence may be about 4 to about 60 J/cm², 4 to about 90 J/cm², 10 to about 90 J/cm², about 10 to about 60 J/cm², about 10 to about 50 J/cm², about 10 to about 40 J/cm², about 10 to about 30 J/cm², about 20 to about 40 J/cm², about 15 J/cm² to 25 J/cm², or about 10 to about 20 J/cm².

In certain embodiments, the biophotonic hydrogel may be re-illuminated at certain intervals. In yet another embodiment, the source of actinic light is in continuous motion over the treated area for the appropriate time of exposure. In yet another embodiment, the biophotonic hydrogel may be illuminated until the biophotonic hydrogel is at least partially photobleached or fully photobleached.

In certain embodiments, the chromophore(s) in the biophotonic hydrogel can be photoexcited by ambient light including from the sun and overhead lighting. In certain embodiments, the chromophore(s) can be photoactivated by light in the visible range of the electromagnetic spectrum. The light can be emitted by any light source such as sunlight, light bulb, an LED device, electronic display screens such as on a television, computer, telephone, mobile device, flashlights on mobile devices. In the methods of the present disclosure, any source of light can be used. For example, a combination of ambient light and direct sunlight or direct artificial light may be used. Ambient light can include overhead lighting such as LED bulbs, fluorescent bulbs, and indirect sunlight.

In the methods of the present disclosure, the biophotonic hydrogel may be removed from the skin following application of light. In other embodiments, the biophotonic hydrogel is left on the tissue for an extended period of time and re-activated with direct or ambient light at appropriate times to treat the condition.

In certain embodiments of the method of the present disclosure, the biophotonic hydrogel can be applied to the tissue, once, twice, three times, four times, five times or six times a week, daily, or at any other frequency. The total treatment time can be one week, two weeks, three weeks, four weeks, five weeks, six weeks, seven weeks, eight weeks, nine weeks, ten weeks, eleven weeks, twelve weeks, or any other length of time deemed appropriate.

In certain embodiments, the biophotonic hydrogel can be used to promote wound healing. In this case, the biophotonic hydrogel may be applied at wound site as deemed appropriate by the physician or other health care providers.

In certain embodiments, the biophotonic hydrogel can be used following wound closure to optimize scar revision. In this case, the biophotonic hydrogel may be applied at regular intervals such as once a week, or at an interval deemed appropriate by the physician or other health care providers.

In certain embodiments, the biophotonic hydrogel can be used following acne treatment to maintain the condition of the treated skin. In this case, the biophotonic hydrogel may be applied at regular intervals such as once a week, or at an interval deemed appropriate by the physician or other health care providers.

In certain embodiments, the biophotonic hydrogel can be used following ablative skin rejuvenation treatment to maintain the condition of the treated skin. In this case, the biophotonic hydrogel may be applied at regular intervals such as once a week, or at an interval deemed appropriate by the physician or other health care providers.

In the methods of the present disclosure, additional components may optionally be included in the biophotonic hydrogel or used in combination with the biophotonic hydrogel. Such additional components include, but are not limited to, healing factors, antimicrobials, oxygen-rich agents, wrinkle fillers such as botox, hyaluronic acid and polylactic acid, fungal, anti-bacterial, anti-viral agents and/or agents that promote collagen synthesis. These additional components may be applied to the skin in a topical fashion, prior to, at the same time of, and/or after topical application of the biophotonic hydrogel of the present disclosure. Suitable healing factors comprise compounds that promote or enhance the healing or regenerative process of the tissues on the application site. During the photoactivation of a biophotonic hydrogel of the present disclosure, there may be an increase of the absorption of molecules of such additional components at the treatment site by the skin or the mucosa. In certain embodiments, an augmentation in the blood flow at the site of treatment can observed for a period of time. An increase in the lymphatic drainage and a possible change in the osmotic equilibrium due to the dynamic interaction of the free radical cascades can be enhanced or even fortified with the inclusion of healing factors. Healing factors may also modulate the biophotonic output from the biophotonic composition such as photobleaching time and profile, or modulate leaching of certain ingredients within the composition. Suitable healing factors include, but are not limited to glucosamines, allantoins, saffron, agents that promote collagen synthesis, anti-fungal, anti-bacterial, anti-viral agents and wound healing factors such as growth factors.

(i) Skin Rejuvenation

The biophotonic hydrogel of the present disclosure may be useful in promoting skin rejuvenation or improving skin condition and appearance. The dermis is the second layer of skin, containing the structural elements of the skin, the connective tissue. There are various types of connective tissue with different functions. Elastin fibers give the skin its elasticity, and collagen gives the skin its strength.

The junction between the dermis and the epidermis is an important structure. The dermal-epidermal junction interlocks forming finger-like epidermal ridges. The cells of the epidermis receive their nutrients from the blood vessels in the dermis. The epidermal ridges increase the surface area of the epidermis that is exposed to these blood vessels and the needed nutrients.

The aging of skin comes with significant physiological changes to the skin. The generation of new skin cells slows down, and the epidermal ridges of the dermal-epidermal junction flatten out. While the number of elastin fibers increases, their structure and coherence decreases. Also the amount of collagen and the thickness of the dermis decrease with the ageing of the skin.

Collagen is a major component of the skin's extracellular matrix, providing a structural framework. During the aging process, the decrease of collagen synthesis and insolubilization of collagen fibers contribute to a thinning of the dermis and loss of the skin's biomechanical properties.

The physiological changes to the skin result in noticeable aging symptoms often referred to as chronological-, intrinsic- and photo-aging. The skin becomes drier, roughness and scaling increase, the appearance becomes duller, and most obviously, fine lines and wrinkles appear. Other symptoms or signs of skin aging include, but are not limited to, thinning and transparent skin, loss of underlying fat (leading to hollowed cheeks and eye sockets as well as noticeable loss of firmness on the hands and neck), bone loss (such that bones shrink away from the skin due to bone loss, which causes sagging skin), dry skin (which might itch), an inability to sweat sufficiently to cool the skin, unwanted facial hair, freckles, age spots, spider veins, rough and leathery skin, fine wrinkles that disappear when stretched, loose skin and/or a blotchy complexion.

The dermal-epidermal junction is a basement membrane that separates the keratinocytes in the epidermis from the extracellular matrix, which lies below in the dermis. This membrane consists of two layers: the basal lamina in contact with the keratinocytes, and the underlying reticular lamina in contact with the extracellular matrix. The basal lamina is rich in collagen type IV and laminin, molecules that play a role in providing a structural network and bioadhesive properties for cell attachment.

Laminin is a glycoprotein that only exists in basement membranes. It is composed of three polypeptide chains (alpha, beta and gamma) arranged in the shape of an asymmetric cross and held together by disulfide bonds. The three chains exist as different subtypes which result in twelve different isoforms for laminin, including Laminin-1 and Laminin-5

The dermis is anchored to hemidesmosomes, specific junction points located on the keratinocytes, which consist of α-integrins and other proteins, at the basal membrane keratinocytes by type VII collagen fibrils. Laminins, and particularly Laminin-5, constitute the real anchor point between hemidesmosomal transmembrane proteins in basal keratinocytes and type VII collagen.

Laminin-5 synthesis and type VII collagen expression have been proven to decrease in aged skin. This causes a loss of contact between dermis and epidermis, and results in the skin losing elasticity and becoming saggy.

Recently another type of wrinkles, generally referred to as expression wrinkles, received general recognition. Expression wrinkles result from a loss of resilience, particularly in the dermis, because of which the skin is no longer able to resume its original state when facial muscles which produce facial expressions.

The biophotonic hydrogels of the present disclosure and methods of the present disclosure may be used to promote skin rejuvenation. In certain embodiments, the biophotonic hydrogels and methods of the present disclosure may be used to promote skin luminosity, reduction of pore size, reducing blotchiness, making even skin tone, reducing dryness, and tightening of the skin, thereby promoting skin rejuvenation. In certain embodiments, the biophotonic hydrogels and methods of the present disclosure promote collagen synthesis. In certain other embodiments, the biophotonic hydrogels and methods of the present disclosure may reduce, diminish, retard or even reverse one or more signs of skin aging including, but not limited to, appearance of fine lines or wrinkles, thin and transparent skin, loss of underlying fat (leading to hollowed cheeks and eye sockets as well as noticeable loss of firmness on the hands and neck), bone loss (such that bones shrink away from the skin due to bone loss, which causes sagging skin), dry skin (which might itch), an inability to sweat sufficiently to cool the skin, unwanted facial hair, freckles, age spots, spider veins, rough and leathery skin, fine wrinkles that disappear when stretched, loose skin, or a blotchy complexion. In certain embodiments, the biophotonic hydrogels and methods of the present disclosure may induce a reduction in pore size, enhance sculpturing of skin subsections, and/or enhance skin translucence.

In certain embodiments, the biophotonic hydrogel may be used in conjunction with collagen promoting agents. Agents that promote collagen synthesis (i.e., pro-collagen synthesis agents) include amino acids, peptides, proteins, lipids, small chemical molecules, natural products and extracts from natural products.

For instance, it was discovered that intake of vitamin C, iron, and collagen can effectively increase the amount of collagen in skin or bone. See, e.g., U.S. Patent Application Publication 2009/0069217. Examples of the vitamin C include an ascorbic acid derivative such as L-ascorbic acid or sodium L-ascorbate, an ascorbic acid preparation obtained by coating ascorbic acid with an emulsifier or the like, and a mixture containing two or more of those vitamin Cs at an arbitrary rate. In addition, natural products containing vitamin C such as acerola or lemon may also be used. Examples of the iron preparation include: an inorganic iron such as ferrous sulfate, sodium ferrous citrate, or ferric pyrophosphate; an organic iron such as heme iron, ferritin iron, or lactoferrin iron; and a mixture containing two or more of those irons at an arbitrary rate. In addition, natural products containing iron such as spinach or liver may also be used. Moreover, examples of the collagen include: an extract obtained by treating bone, skin, or the like of a mammal such as bovine or swine with an acid or alkaline; a peptide obtained by hydrolyzing the extract with a protease such as pepsin, trypsin, or chymotrypsin; and a mixture containing two or more of those collagens at an arbitrary rate. Collagens extracted from plant sources may also be used.

Additional pro-collagen synthesis agents are described, for example, in U.S. Pat. Nos. 7,598,291; 7,722,904; 6,203,805; 5,529,769; and U.S. Patent Application Publications Nos: 2006/0247313; 2008/0108681; 2011/0130459; 2009/0325885; and 2011/0086060.

(ii) Skin disorders

The biophotonic hydrogels and methods of the present disclosure may be used to treat skin disorders that include, but are not limited to, erythema, telangiectasia, actinic telangiectasia, basal cell carcinoma, contact dermatitis, dermatofibrosarcoma protuberans, genital warts, hidradenitis suppurativa, melanoma, merkel cell carcinoma, nummular dermatitis, molloscum contagiosum, psoriasis, psoriatic arthritis, rosacea, scabies, scalp psoriasis, sebaceous carcinoma, squamous cell carcinoma, seborrheic dermatitis, seborrheic keratosis, shingles, tinea versicolor, warts, skin cancer, pemphigus, sunburn, dermatitis, eczema, rashes, impetigo, lichen simplex chronicus, rhinophyma, perioral dermatitis, pseudofolliculitis barbae, drug eruptions, erythema multiforme, erythema nodosum, granuloma annulare, actinic keratosis, purpura, alopecia areata, aphthous stomatitis, dry skin, chapping, xerosis, ichthyosis vulgaris, fungal infections, herpes simplex, intertrigo, keloids, keratoses, milia, moluscum contagiosum, pityriasis rosea, pruritus, urticaria, and vascular tumors and malformations. Dermatitis includes contact dermatitis, atopic dermatitis, seborrheic dermatitis, nummular dermatitis, generalized exfoliative dermatitis, and statis dermatitis. Skin cancers include melanoma, basal cell carcinoma, and squamous cell carcinoma.

(iii) Acne and Acne Scars

The biophotonic hydrogels and methods of the present disclosure may be used to treat acne. As used herein, “acne” means a disorder of the skin caused by inflammation of skin glands or hair follicles. The biophotonic hydrogels and methods of the disclosure can be used to treat acne at early pre-emergent stages or later stages where lesions from acne are visible.

Mild, moderate and severe acne can be treated with embodiments of biophotonic hydrogels and methods. Early pre-emergent stages of acne usually begin with an excessive secretion of sebum or dermal oil from the sebaceous glands located in the pilosebaceous apparatus. Sebum reaches the skin surface through the duct of the hair follicle. The presence of excessive amounts of sebum in the duct and on the skin tends to obstruct or stagnate the normal flow of sebum from the follicular duct, thus producing a thickening and solidification of the sebum to create a solid plug known as a comedone. In the normal sequence of developing acne, hyperkeratinazation of the follicular opening is stimulated, thus completing blocking of the duct. The usual results are papules, pustules, or cysts, often contaminated with bacteria, which cause secondary infections. Acne is characterized particularly by the presence of comedones, inflammatory papules, or cysts. The appearance of acne may range from slight skin irritation to pitting and even the development of disfiguring scars. Accordingly, the biophotonic hydrogels and methods of the present disclosure can be used to treat one or more of skin irritation, pitting, development of scars, comedones, inflammatory papules, cysts, hyperkeratinazation, and thickening and hardening of sebum associated with acne.

Some skin disorders present various symptoms including redness, flushing, burning, scaling, pimples, papules, pustules, comedones, macules, nodules, vesicles, blisters, telangiectasia, spider veins, sores, surface irritations or pain, itching, inflammation, red, purple, or blue patches or discolorations, moles, and/or tumors.

The biophotonic hydrogels and methods of the present disclosure may be used to treat various types of acne. Some types of acne include, for example, acne vulgaris, cystic acne, acne atrophica, bromide acne, chlorine acne, acne conglobata, acne cosmetica, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, and nodulocystic acne.

In certain embodiments, the biophotonic hydrogel of the present disclosure is used in conjunction with systemic or topical antibiotic treatment. For example, antibiotics used to treat acne include tetracycline, erythromycin, minocycline, doxycycline, which may also be used with the compositions and methods of the present disclosure. The use of the biophotonic hydrogel can reduce the time needed for the antibiotic treatment or reduce the dosage.

(iv) Wound Healing

The biophotonic hydrogels and methods of the present disclosure may be used to treat wounds, promote wound healing, promote tissue repair and/or prevent or reduce cosmesis including improvement of motor function (e.g. movement of joints). Wounds that may be treated by the biophotonic hydrogels and methods of the present disclosure include, for example, injuries to the skin and subcutaneous tissue initiated in different ways (e.g., pressure ulcers from extended bed rest, wounds induced by trauma or surgery, burns, ulcers linked to diabetes or venous insufficiency, wounds induced by conditions such as periodontitis) and with varying characteristics. In certain embodiments, the present disclosure provides biophotonic hydrogels and methods for treating and/or promoting the healing of, for example, burns, incisions, excisions, lesions, lacerations, abrasions, puncture or penetrating wounds, surgical wounds, contusions, hematomas, crushing injuries, amputations, sores and ulcers.

The biophotonic hydrogels and methods of the present disclosure may be used to treat and/or promote the healing of chronic cutaneous ulcers or wounds, which are wounds that have failed to proceed through an orderly and timely series of events to produce a durable structural, functional, and cosmetic closure. The vast majority of chronic wounds can be classified into three categories based on their etiology: pressure ulcers, neuropathic (diabetic foot) ulcers and vascular (venous or arterial) ulcers.

For example, the present disclosure provides the biophotonic hydrogels and methods for treating and/or promoting healing of a diabetic ulcer. Diabetic patients are prone to foot and other ulcerations due to both neurologic and vascular complications. Peripheral neuropathy can cause altered or complete loss of sensation in the foot and/or leg. Diabetic patients with advanced neuropathy lose all ability for sharp-dull discrimination. Any cuts or trauma to the foot may go completely unnoticed for days or weeks in a patient with neuropathy. A patient with advanced neuropathy loses the ability to sense a sustained pressure insult, as a result, tissue ischemia and necrosis may occur leading to for example, plantar ulcerations. Microvascular disease is one of the significant complications for diabetics which may also lead to ulcerations. In certain embodiments, the biophotonic hydrogels and methods of treating a chronic wound are provided herein, where the chronic wound is characterized by diabetic foot ulcers and/or ulcerations due to neurologic and/or vascular complications of diabetes.

In other examples, the present disclosure provides biophotonic hydrogels and methods for treating and/or promoting healing of a pressure ulcer. Pressure ulcers include bed sores, decubitus ulcers and ischial tuberosity ulcers and can cause considerable pain and discomfort to a patient. A pressure ulcer can occur as a result of a prolonged pressure applied to the skin. Thus, pressure can be exerted on the skin of a patient due to the weight or mass of an individual. A pressure ulcer can develop when blood supply to an area of the skin is obstructed or cut off for more than two or three hours. The affected skin area can turn red, become painful and necrotic. If untreated, the skin can break open and become infected. A pressure ulcer is therefore a skin ulcer that occurs in an area of the skin that is under pressure from e.g. lying in bed, sitting in a wheelchair, and/or wearing a cast for a prolonged period of time. Pressure ulcers can occur when a person is bedridden, unconscious, unable to sense pain, or immobile. Pressure ulcers often occur in boney prominences of the body such as the buttocks area (on the sacrum or iliac crest), or on the heels of foot.

Additional types of wounds that can be treated by the biophotonic hydrogels and methods of the present disclosure include those disclosed by U.S. Pat. Appl. Publ. No. 2009/0220450.

There are three distinct phases in the wound healing process. First, in the inflammatory phase, which typically occurs from the moment a wound occurs until the first two to five days, platelets aggregate to deposit granules, promoting the deposit of fibrin and stimulating the release of growth factors. Leukocytes migrate to the wound site and begin to digest and transport debris away from the wound. During this inflammatory phase, monocytes are also converted to macrophages, which release growth factors for stimulating angiogenesis and the production of fibroblasts.

Second, in the proliferative phase, which typically occurs from two days to three weeks from wound occurrence, granulation tissue forms, and epithelialization and contraction begin. Fibroblasts, which are key cell types in this phase, proliferate and synthesize collagen to fill the wound and provide a strong matrix on which epithelial cells grow. As fibroblasts produce collagen, vascularization extends from nearby vessels, resulting in granulation tissue. Granulation tissue typically grows from the base of the wound. Epithelialization involves the migration of epithelial cells from the wound surfaces to seal the wound. Epithelial cells are driven by the need to contact cells of like type and are guided by a network of fibrin strands that function as a grid over which these cells migrate. Contractile cells called myofibroblasts appear in wounds, and aid in wound closure. These cells exhibit collagen synthesis and contractility, and are common in granulating wounds.

Third, in the remodeling phase, the final phase of wound healing which can take place from three weeks up to several years from wound occurrence, collagen in the scar undergoes repeated degradation and re-synthesis. During this phase, the tensile strength of the newly formed skin increases.

However, as the rate of wound healing increases, there is often an associated increase in scar formation. Scarring is a consequence of the healing process in most adult animal and human tissues. Scar tissue is not identical to the tissue which it replaces, as it is usually of inferior functional quality. The types of scars include, but are not limited to, atrophic, hypertrophic and keloidal scars, as well as scar contractures. Atrophic scars are flat and depressed below the surrounding skin as a valley or hole. Hypertrophic scars are elevated scars that remain within the boundaries of the original lesion, and often contain excessive collagen arranged in an abnormal pattern. Keloidal scars are elevated scars that spread beyond the margins of the original wound and invade the surrounding normal skin in a way that is site specific, and often contain whorls of collagen arranged in an abnormal fashion.

In contrast, normal skin consists of collagen fibers arranged in a basket-weave pattern, which contributes to both the strength and elasticity of the dermis. Thus, to achieve a smoother wound healing process, an approach is needed that not only stimulates collagen production, but also does so in a way that reduces scar formation.

The biophotonic hydrogels and methods of the present disclosure promote the wound healing by promoting the formation of substantially uniform epithelialization; promoting collagen synthesis; promoting controlled contraction; and/or by reducing the formation of scar tissue. In certain embodiments, the biophotonic hydrogels and methods of the present disclosure may promote wound healing by promoting the formation of substantially uniform epithelialization. In some embodiments, the biophotonic hydrogels and methods of the present disclosure promote collagen synthesis. In some other embodiments, the biophotonic hydrogels and methods of the present disclosure promote controlled contraction. In certain embodiments, the biophotonic hydrogels and methods of the present disclosure promote wound healing, for example, by reducing the formation of scar tissue.

In the methods of the present disclosure, the biophotonic hydrogels of the present disclosure may also be used in combination with negative pressure assisted wound closure devices and systems.

In certain embodiments, the biophotonic hydrogel is kept in place for up to one, two or 3 weeks, and illuminated with light which may include ambient light at various intervals. In this case, the composition may be covered up in between exposure to light with an opaque material or left exposed to light.

### (6) Kits

The present disclosure also provides kits for preparing a biophotonic material and/or providing any of the components required for forming biophotonic materials of the present disclosure.

In some embodiments, the kit includes containers comprising the components or compositions that can be used to make the biophotonic hydrogels of the present disclosure. In some embodiments, the kit includes biophotonic hydrogel material of the present disclosure. The different components making up the biophotonic hydrogel materials of the present disclosure may be provided in separate containers. For example, the HEAA polymerisable monomer may be provided in a container separate from the chromophore. Examples of such containers are dual chamber syringes, dual chamber containers with removable partitions, sachets with pouches, and multiple-compartment blister packs. Another example is one of the components being provided in a syringe which can be injected into a container of another component.

In other embodiments, the kit comprises a systemic drug for augmenting the treatment of the biophotonic hydrogels of the present disclosure. For example, the kit may include a systemic or topical antibiotic, hormone treatment (e.g. for acne treatment or wound healing), or a negative pressure device.

In other embodiments, the kit comprises a means for applying the components of the biophotonic hydrogel materials.

In certain aspects, there is provided a container comprising a chamber for holding a biophotonic hydrogel material, and an outlet in communication with the chamber for discharging the biophotonic material from the container, wherein the biophotonic material comprises at least one chromophore.

In certain embodiments of the kit, the kit may further comprise a light source such as a portable light with a wavelength appropriate to activate the chromophore of the biophotonic hydrogel. The portable light may be battery operated or re-chargeable.

Written instructions on how to use the forming biophotonic hydrogels in accordance with the present disclosure may be included in the kit, or may be included on or associated with the containers comprising the compositions or components making up the biophotonic hydrogel materials of the present disclosure.

Identification of equivalent biophotonic hydrogels, methods and kits are well within the skill of the ordinary practitioner and would require no more than routine experimentation, in light of the teachings of the present disclosure.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein. All references cited herein are incorporated by reference in their entirety and made part of this application.

Practice of the disclosure will be still more fully understood from the following examples, which are presented herein for illustration only and should not be construed as limiting the disclosure in any way.

### Examples

### Example 1: Hydrogel of poly(hydroxyethyl acrylamide)

An aqueous solution containing 2.025 g of HEAA (monomer), 0.274 g of PEGDA (cross-linker), 0.048 g of TEA (initiator) and 7.50 mL of H₂O was prepared at room temperature. This solution was added with 0.1 mL of Eosin Y solution (10.9 mg/mL), 0.1 mL of fluorescein solution (10.9 mg/mL) and 0.1 mL of NVP solution (0.411 g/mL). The final concentration of Eosin Y in the hydrogel was 109 microgram per gram of hydrogel. Then the resulting mixture was vigorously homogenised and casted into petri dishes to obtain stiff hydrogels with a thickness of about 2 mm after illumination with blue light (peak wavelength between 400-470 nm and a power density of about 30-150 mW/cm²) for 2 minutes.

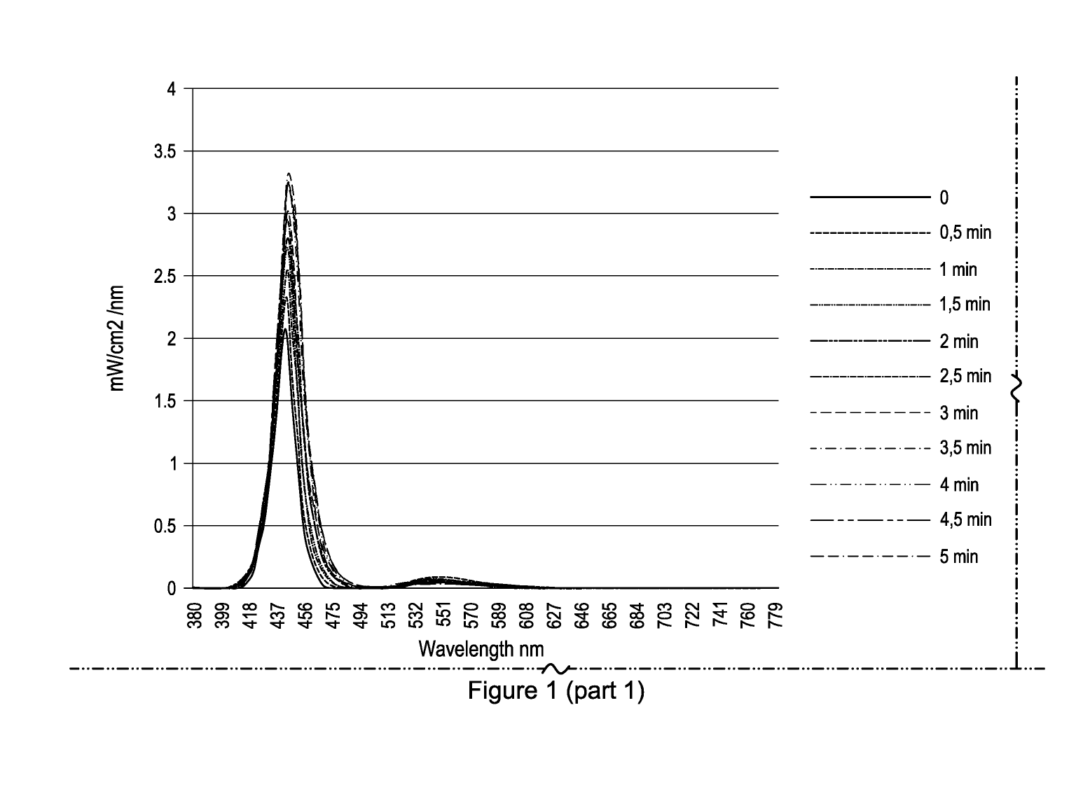

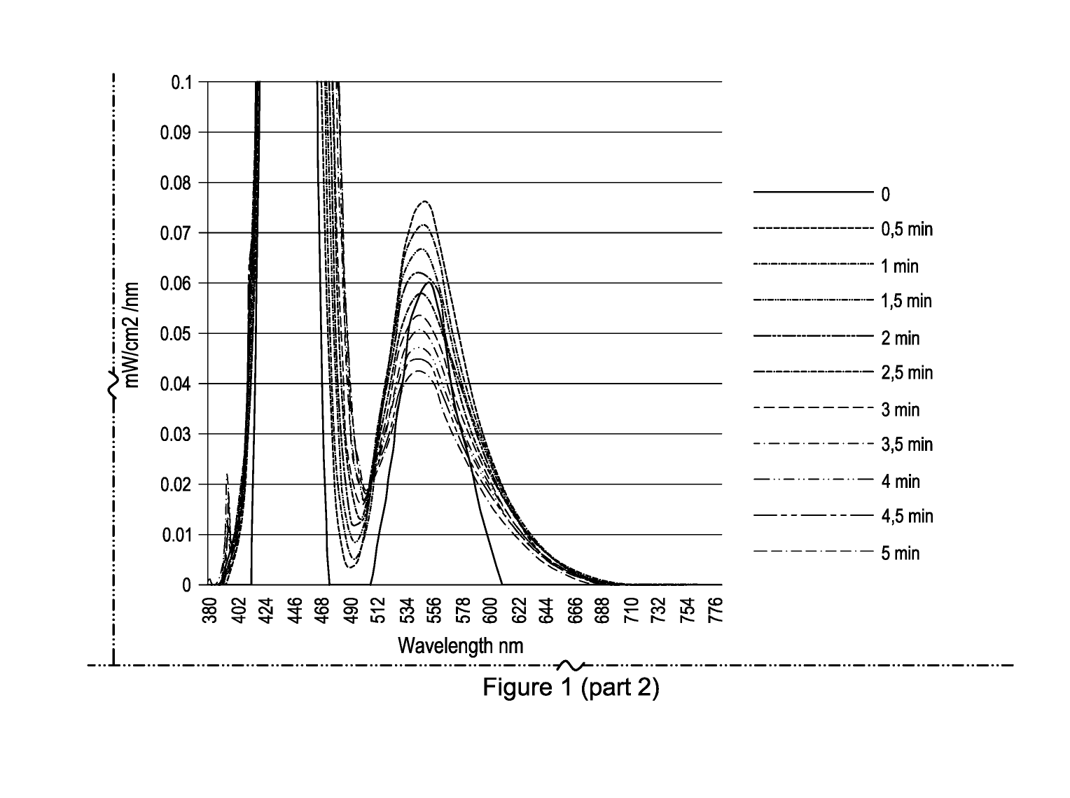

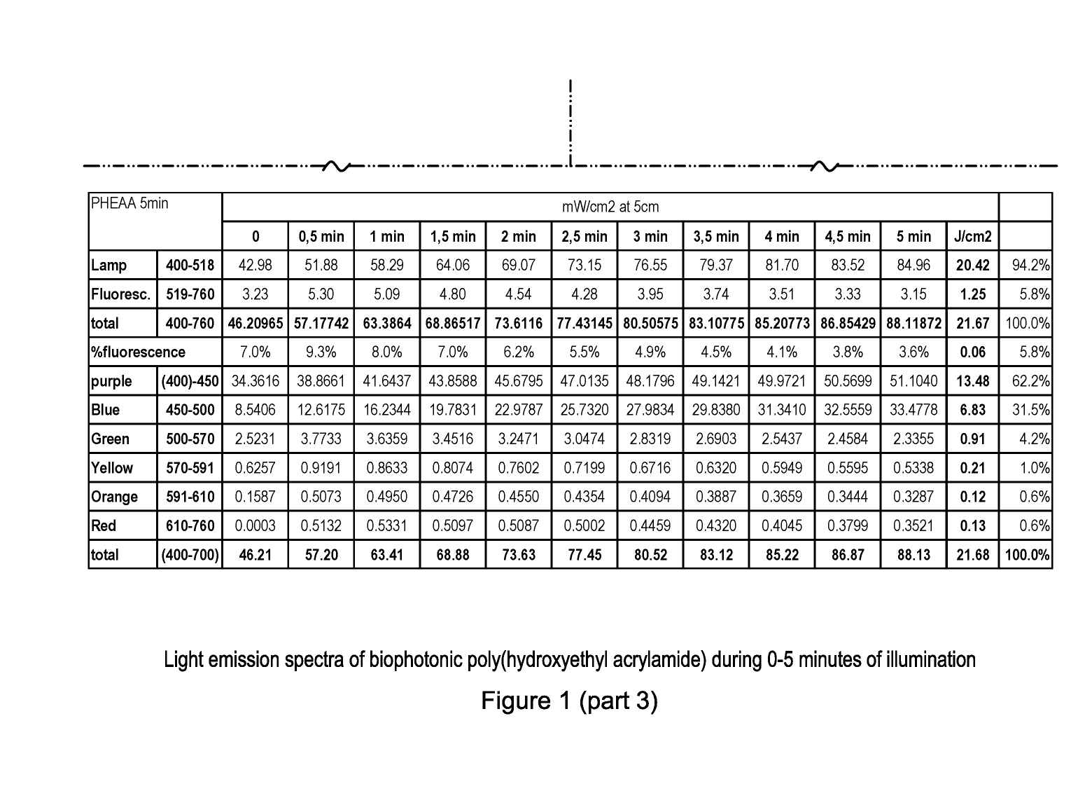

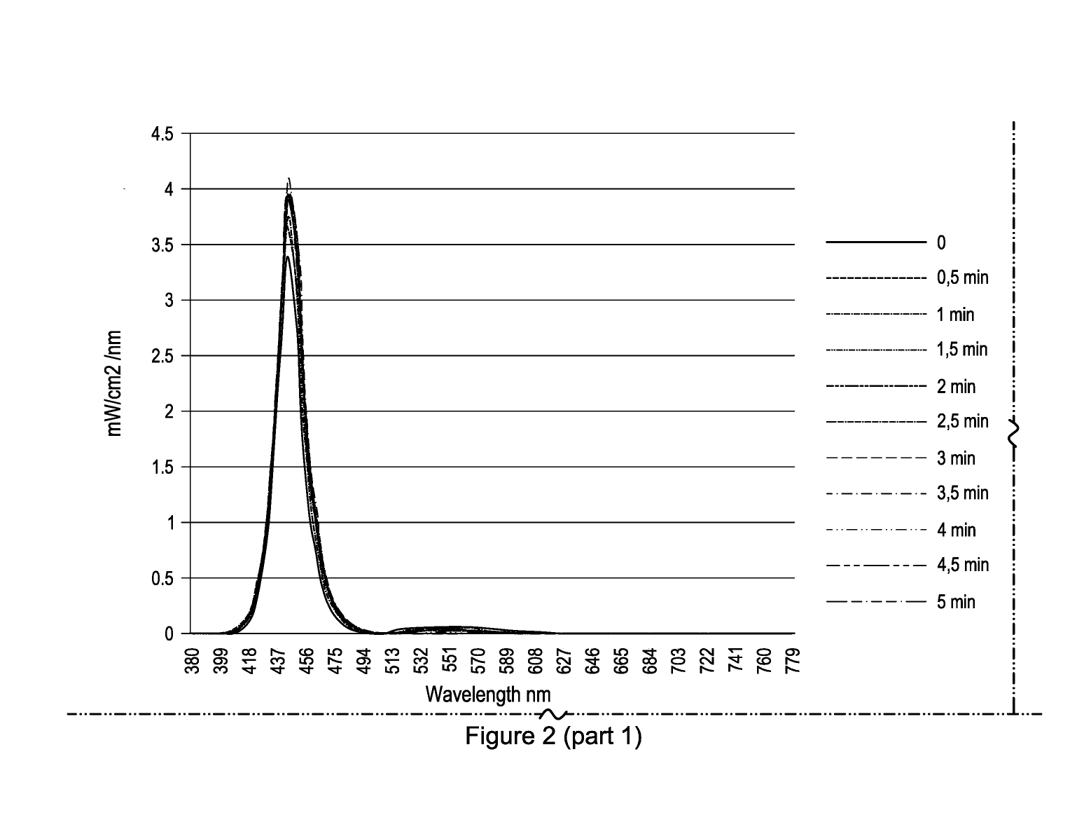

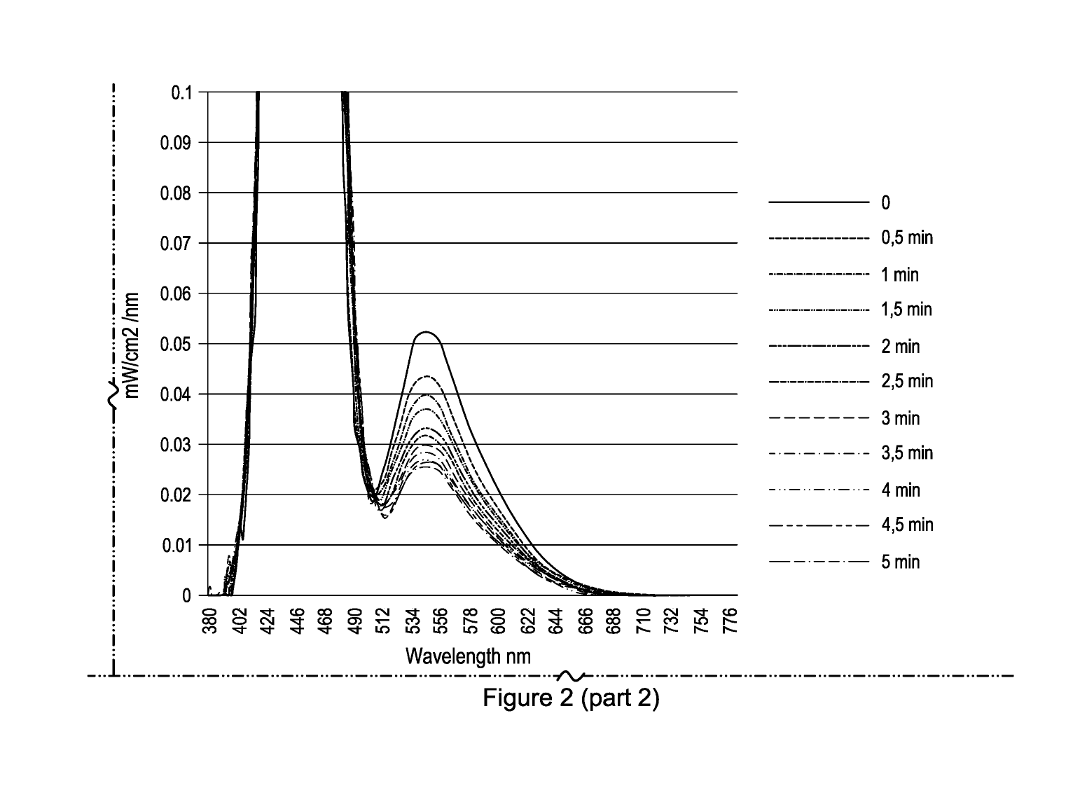

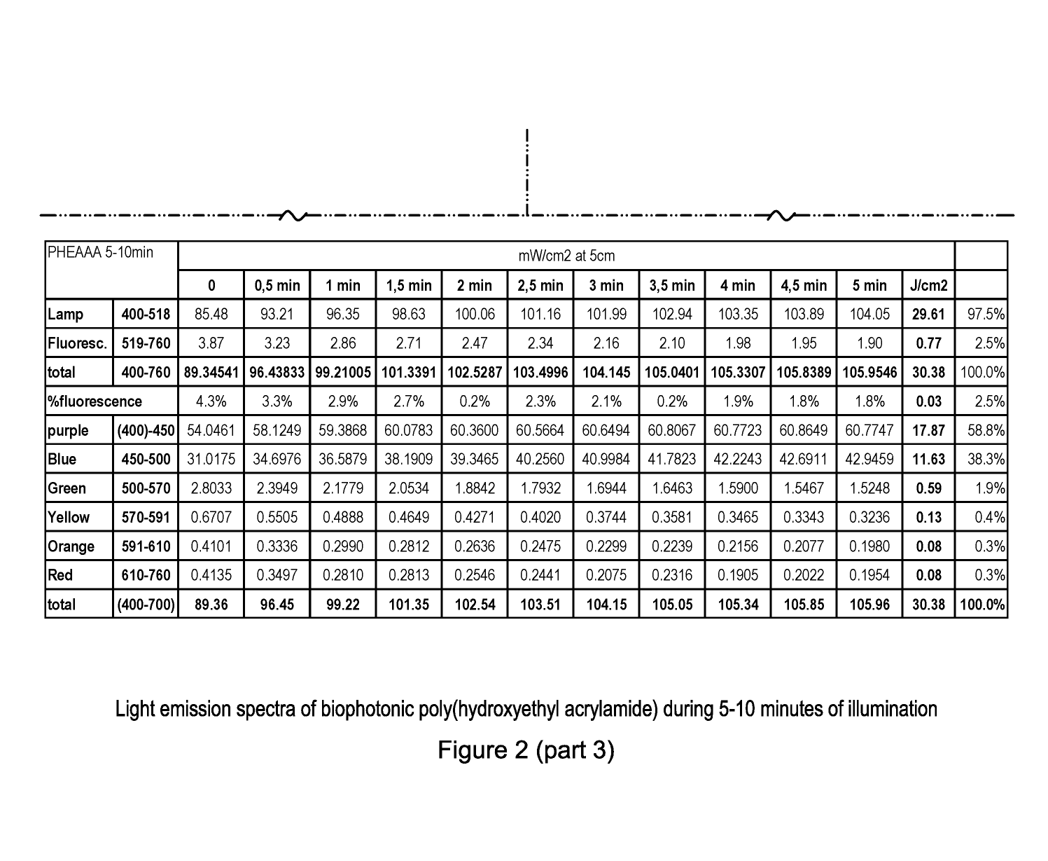

Light emitted through and by the membrane was measured using a SP-100 spectroradiometer (SP-100, ORB Optronix) whilst being illuminated with light having a peak emission wavelength of 450 nm (peak wavelength ranging between 400-470 nm and a power density of about 30-150 mW/cm²) for 5 minutes. FIGS. 1 and 2 show the emission spectra from the membrane after 5 and 10 minutes of illumination respectively. As can be seen, despite the loss in fluorescence, the chromophores did not fully photobleach after 10 minutes of illumination. The biophotonic membrane retained about 35% of its initial fluorescence activity.

### Example 2: Hydrogel of poly(hydroxyethyl acrylamide)/Gelatin

In this experiment, 0.250 g of Gelatin was dissolved in 8.00 mL of H₂O previously warmed to around 40° C. Then, 2.024 g of HEAA, 0.253 g of PEGDA and 0.034 g of TEA were added to the gelatin solution and the mixture was left under stirring for about 15 minutes at room temperature. While maintaining stirring, 0.10 mL of Eosin Y solution (10.9 mg/mL), 0.10 mL of fluorescein solution (10.9 mg/mL) and 0.10 mL of NVP solution were added to the resulting solution. Once homogenised, the solution was casted in petri dishes and illuminated during 2 minutes with blue light, as before, to photoinitiate polymerisation and cross-linking to form hydrogels incorporating chromophores. Here also, the casted volume was such as the thickness of the hydrogel is around 2 mm.

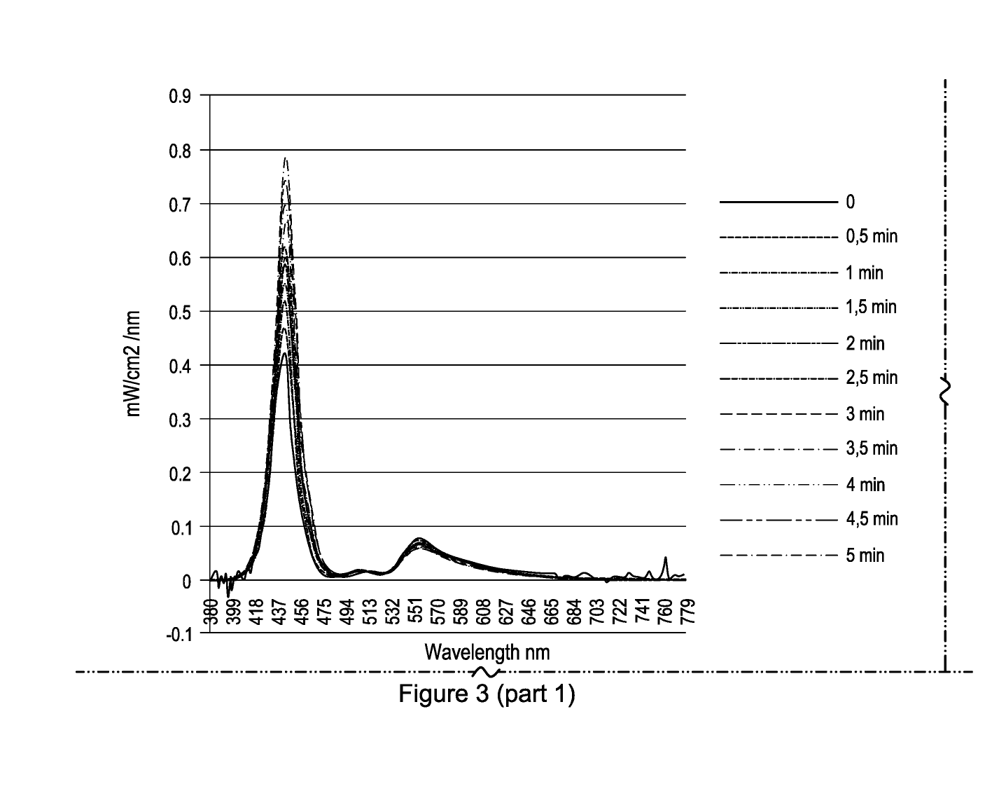

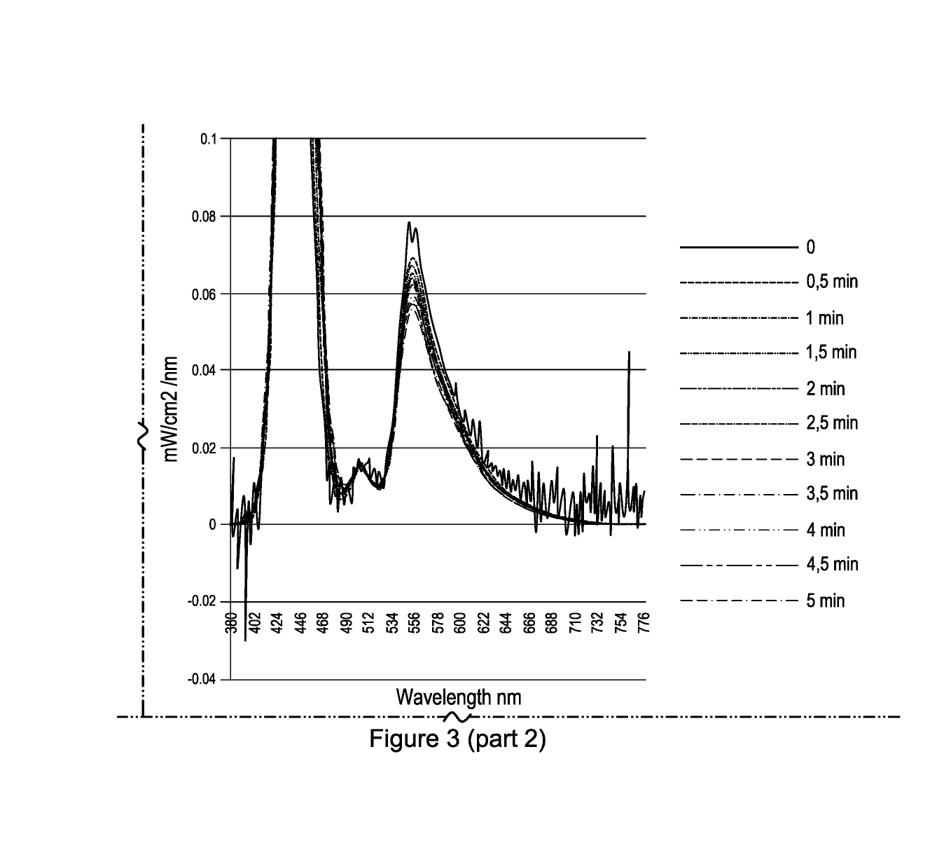

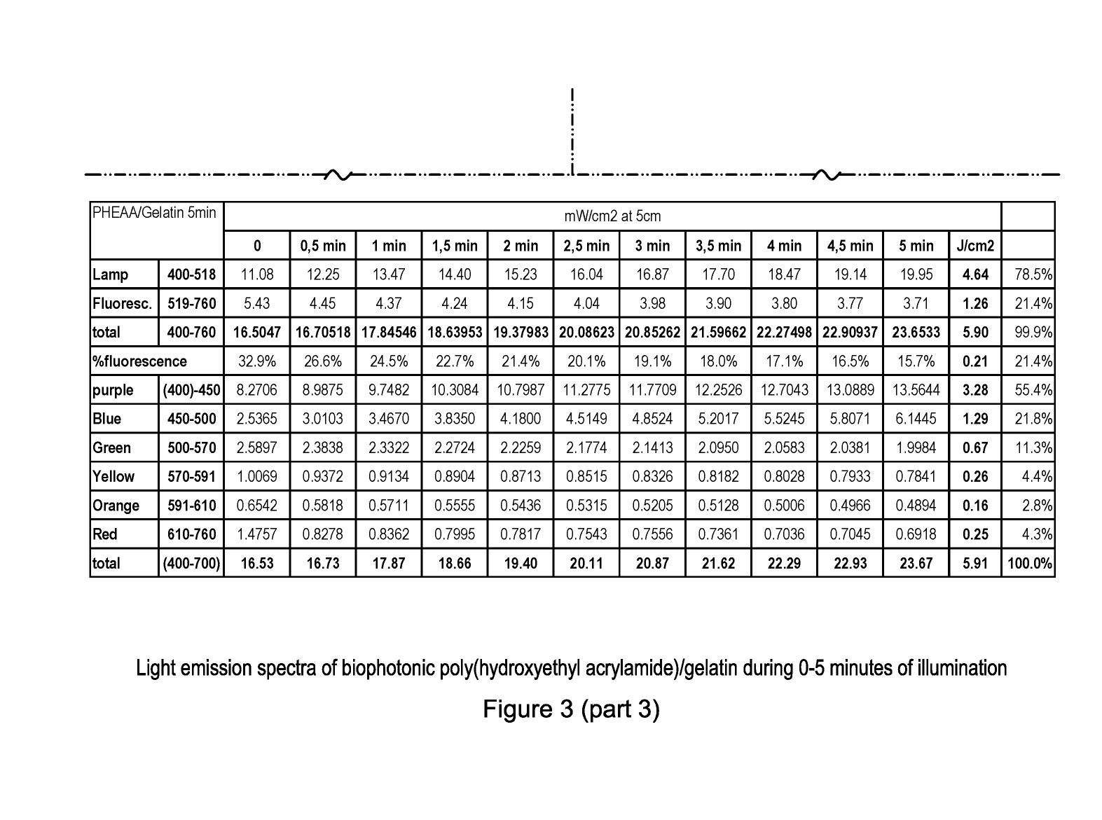

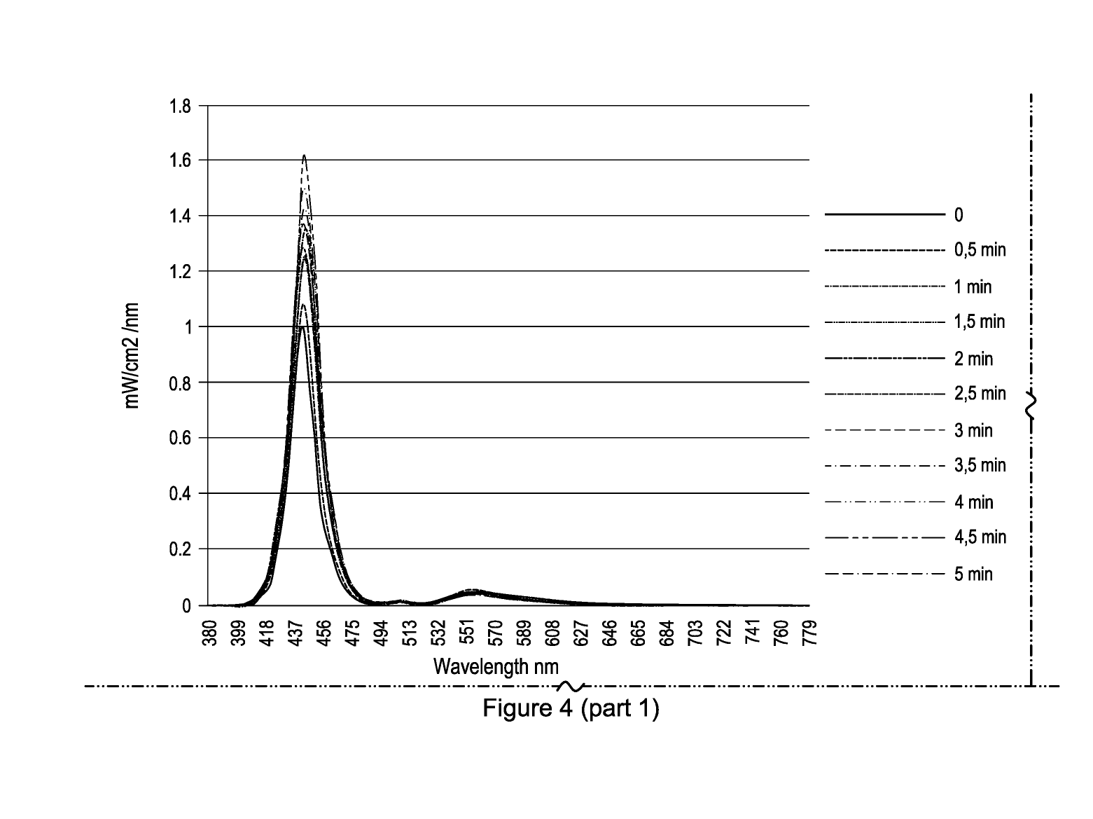

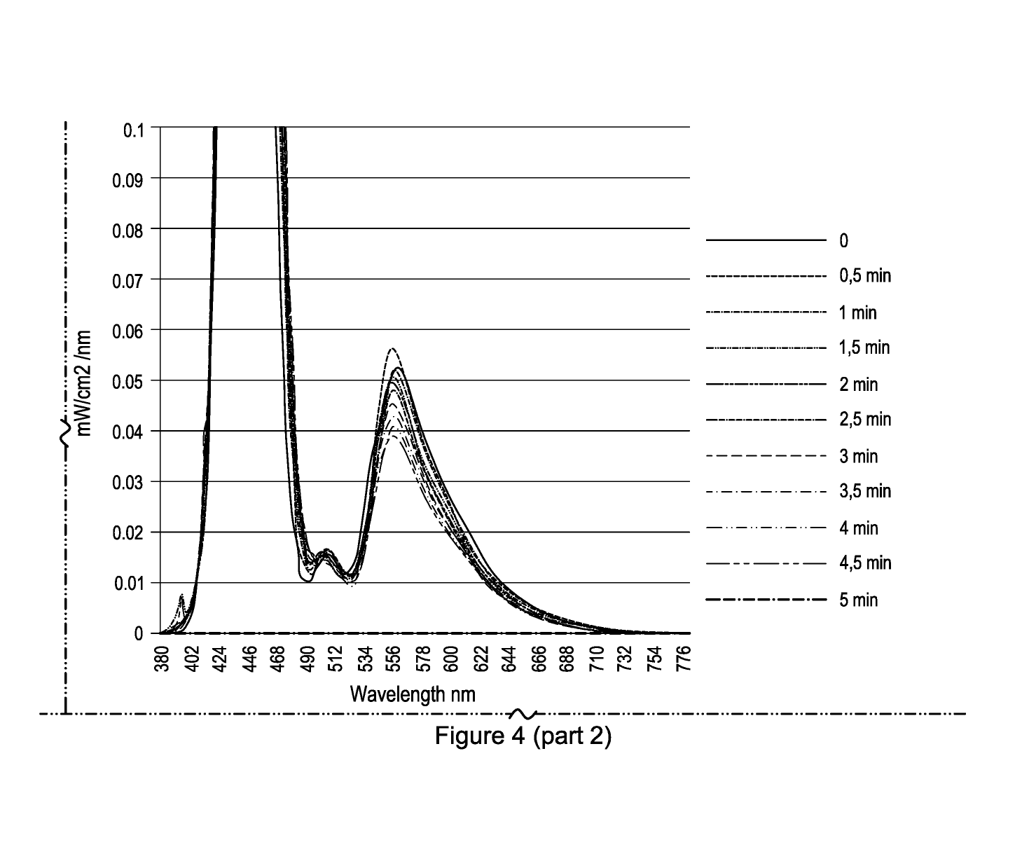

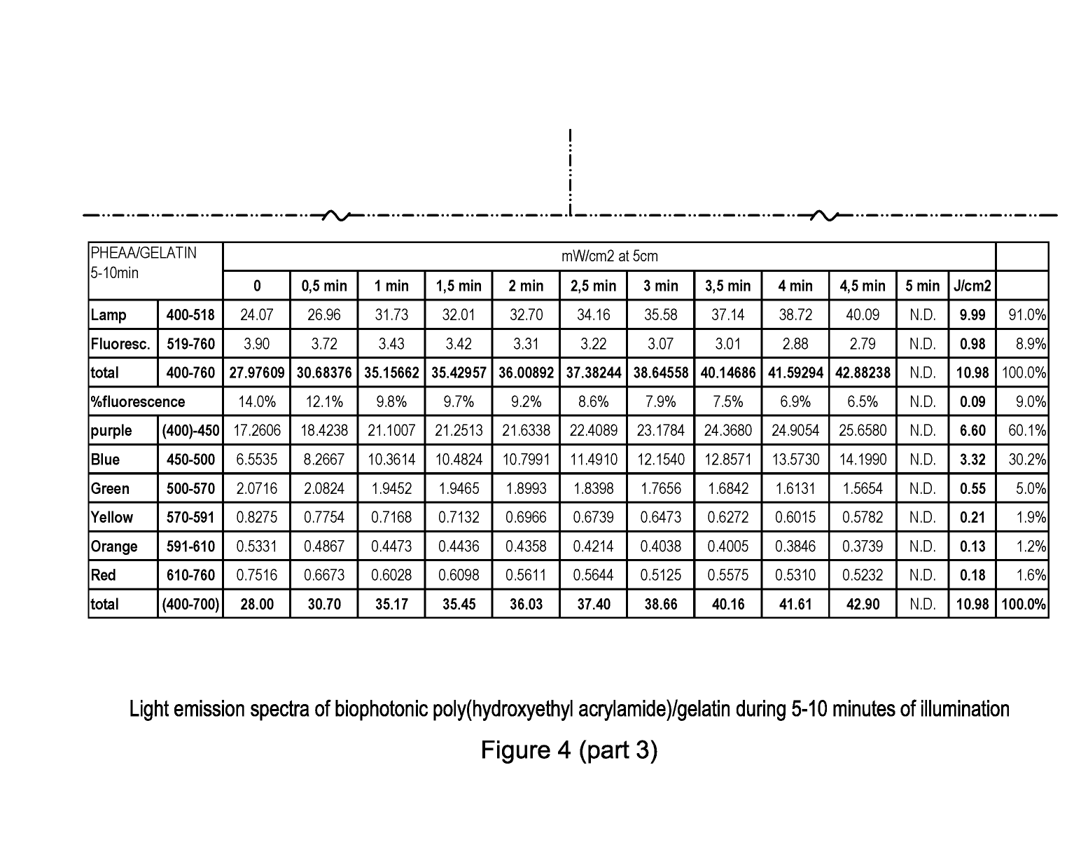

Activating blue light transmitted through the polymer and fluorescent light emitted from the polymer was measured as in Example 1. FIG. 3, which displays the emission spectrum after exposure of the biophotonic membrane to the blue light during 5 minutes, revealed a partial photobleaching of the chromophores. It can be estimated that the membrane lost approximately 32% of its initial fluorescence activity after 5 minutes of illumination and about 50% after 10 minutes of illumination (See FIG. 4).

### Example 3: Hydrogel of poly(hydroxyethyl acrylamide)/HEC

Amounts of 2.007 g of HEAA, 0.256 g of PEGDA and 0.030 g of TEA were added and thoroughly mixed to 7.522 g of aqueous solution (2%) of hydroxyethyl cellulose (HEC). To the resulting solution, 0.10 mL of Eosin Y solution (10.9 mg/mL), 0.10 mL of fluorescein solution (10.9 mg/mL) and 0.10 mL of NVP solution were added and homogenised to obtain photoactive solution. Then the solution was casted into petri dishes and exposed to blue light in order to photoinitiate polymerisation/crosslinking and form hydrogels containing chromophores after 2 minute light exposure as before.

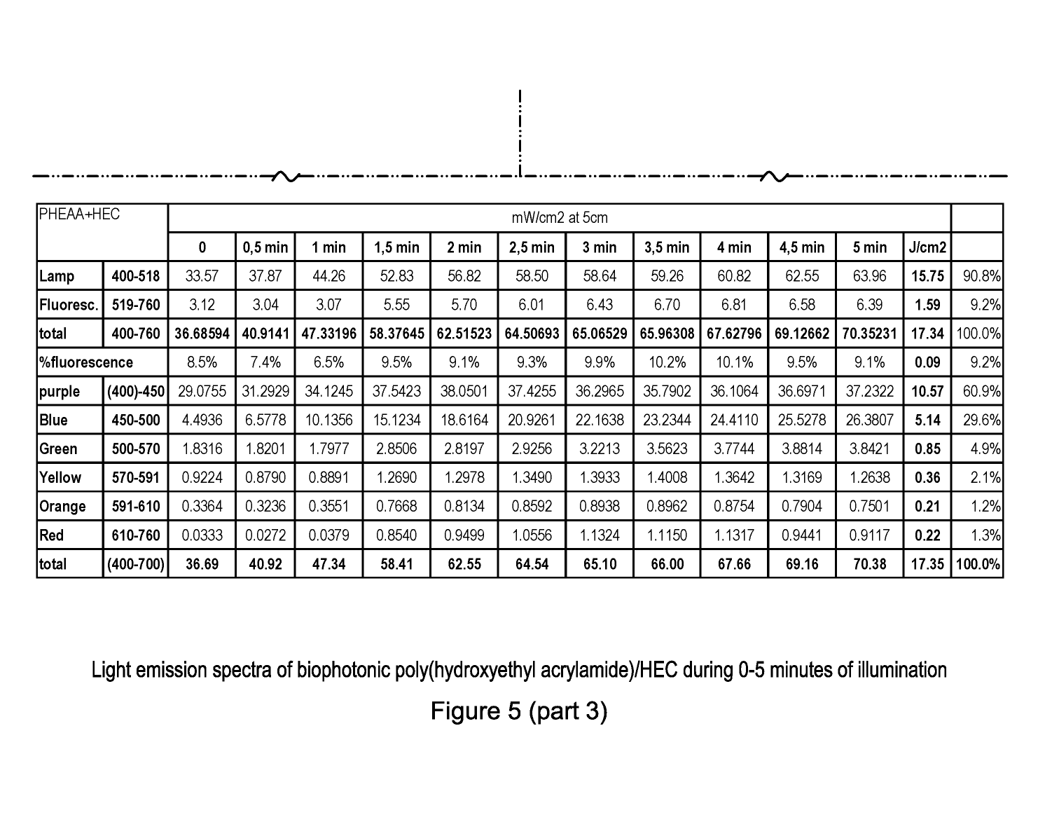

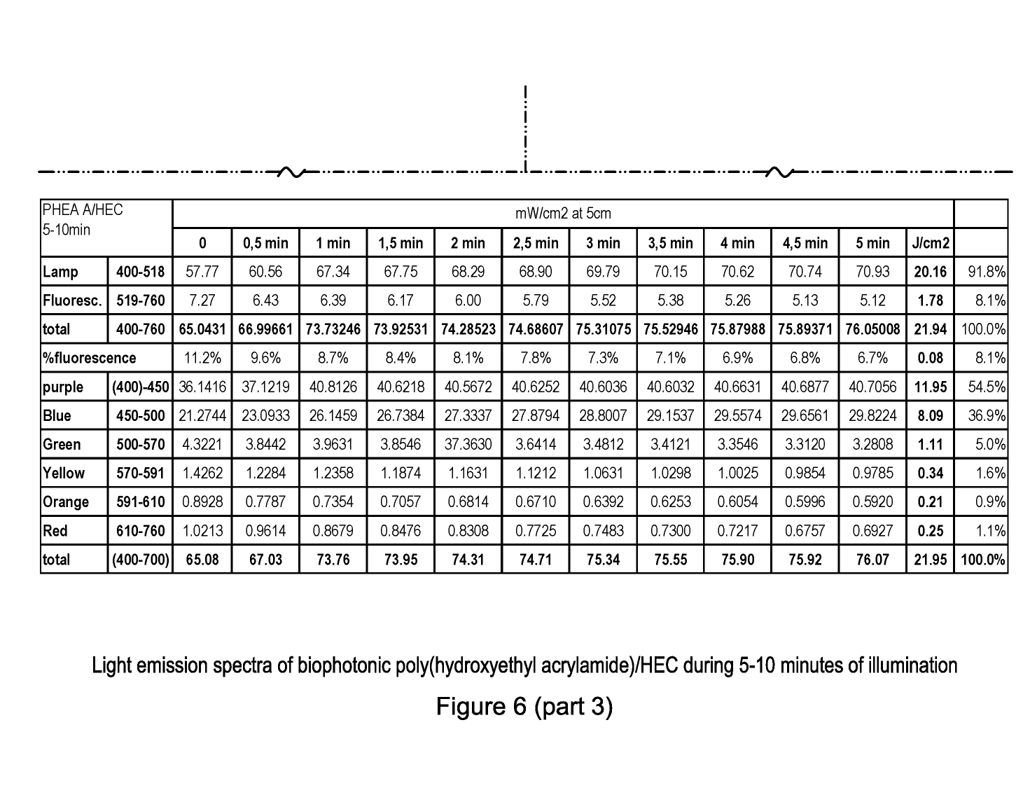

Activating blue light transmitted through the polymer and fluorescent light emitted from the polymer was measured as in Example 1. FIGS. 5 and 6 show the spectra of light detected beneath the biophotonic membrane during 5 and 10 minutes respectively of light activation. Surprisingly, the data indicates a significant increase in the fluorescence during the first 5 minutes of illumination. At 5 minutes the measured intensity of fluorescence was more than twice that measured initially. Also, this increase in fluorescence was associated with clear coloration change of the biophotonic membrane, from pinkish to yellow, suggesting nearly complete photobleaching of eosin. Between 5 and 10 minutes of illumination, a slight decrease in the fluorescence was observed ending with a fluorescence approximately 160% higher than that recorded at time zero.

### Example 4: Hydrogel of poly(hydroxyethyl acrylamide)/PL-F127

Amounts of 2.002 g of HEAA, 0.240 g of PEGDA and 0.035 g of TEA were added to 7.512 g of aqueous solution of thermosetting pluronic PL-F127 (25%). The mixture was homogenised by vigorous stirring, and while maintaining stirring the resulting solution was added with 0.1 mL of Eosin Y solution (10.9 mg/mL), 0.1 mL of fluorescein solution (10.9 mg/mL) and 0.1 mL of NVP solution. Then, the resulting mixture was casted in petri dishes and illuminated by the blue light of Example 1 for 2 minutes to form poly(hydroxyethyl acrylamide)/PL-F127 cross-linked with PEGDA. The volume casted was controlled such as the thickness of the formed hydrogel was around 2 mm.

Light emitted through the polymer and fluorescence emitted by the polymer was measured using a SP-100 spectroradiometer (SP-100, ORB Optronix).

FIG. 7 shows the light spectrum recorded during exposure of the biophotonic membrane to blue light for 5 minutes. As can be seen, the emitted fluorescence in this case was significantly higher than that observed in the previous Examples, although all the membranes contained the same concentration of eosin y and fluorescein. While not being bound to theory, it is thought that this fluorescence enhancement can be attributed to the surfactant nature of Pluronic F-127.

### Example 5: Hydrogel of poly(hydroxyethyl acrylamide)/PL-F127-CTAB

Amounts of 2.010 g of HEAA, 0.499 g of PEGDA, 0.081 g of cetyltrimethyl ammonium bromide (CTAB) and 0.0453 g of TEA were added to 8.00 g of aqueous solution of thermosetting pluronic PL-F127 (25%). The mixture was homogenized by vigorous stirring, and while maintaining stirring the resulting solution was added with 0.1 mL of Eosin Y solution (10.9 mg/mL), 0.1 mL of fluorescein solution (10.9 mg/mL) and 0.1 mL of NVP solution. Then, the resulting mixture was casted in petri dishes and illuminated by the blue light of Example 1 for 30 seconds to form poly(hydroxyethyl acrylamide)/PL-F127-CTAB cross-linked with PEGDA. The volume casted was controlled such as the thickness of the formed hydrogel was around 2 mm.

Light emitted through the polymer and fluorescence emitted by the polymer was measured using a SP-100 spectroradiometer (SP-100, ORB Optronix).

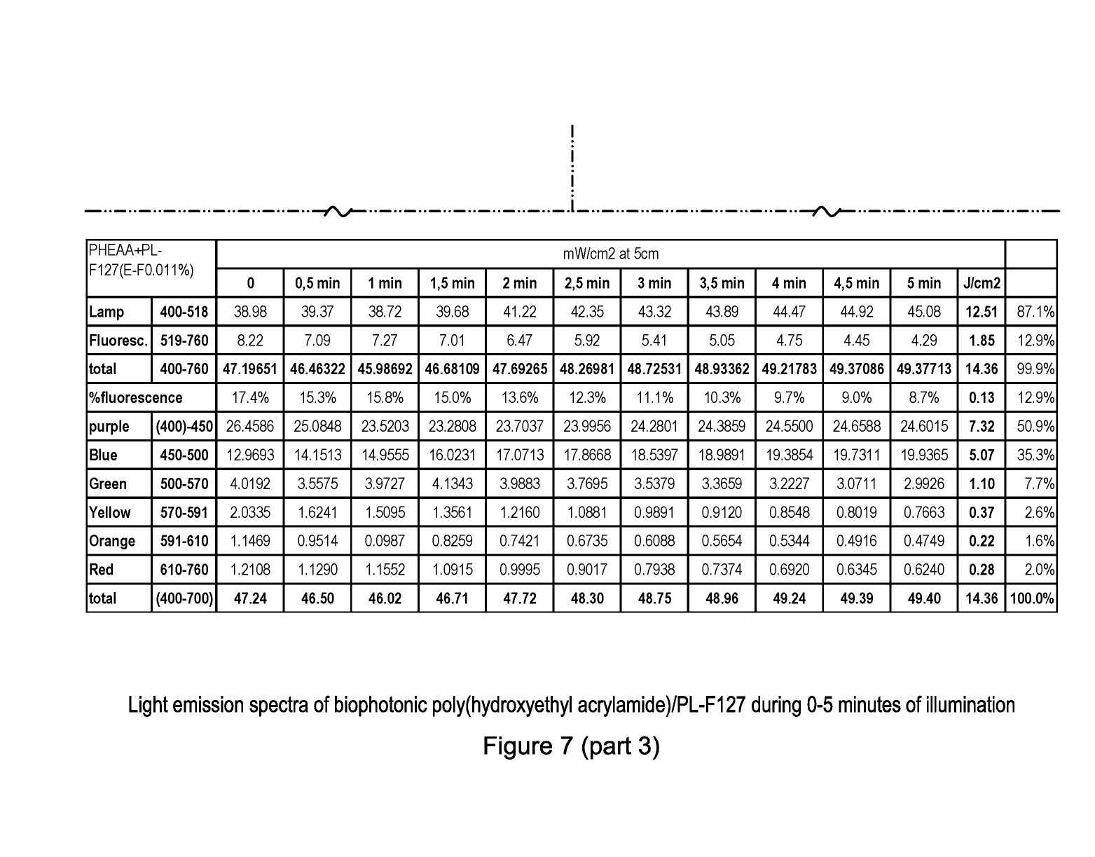

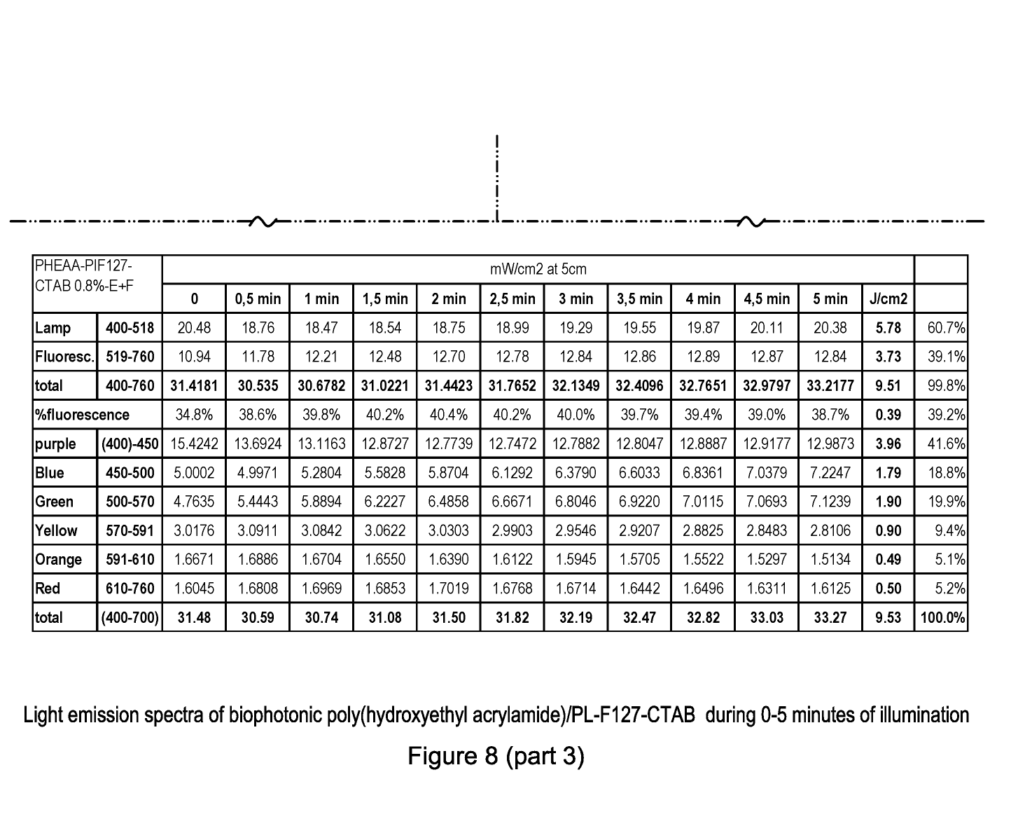

FIG. 8 shows the light spectrum recorded during exposure of the biophotonic membrane to blue light for 5 minutes. As can be seen, the emitted fluorescence in this case was significantly higher than that observed in the previous Examples, although all the membranes contained the same concentration of Eosin Y and fluorescein. In comparison to the emitted fluorescence exhibited by the membrane observed in Example 4, the membrane in this Example 5 exhibited a decreased amount of purple and blue light being emitted, and with respect to the green, orange and red light emitted an approximate doubling of each of these respective light colors, and with respect to the amount of yellow light emitted from the biophotonic hydrogel of Example 5, an approximate tripling of this color of light being emitted.

### Example 6: Hydrogel of poly(hydroxyethyl acrylamide)/PL-F127-Benonite

Amounts of 2.010 g of HEAA, 0.537 g of PEGDA, 0.021 g of bentonite (B) and 0.0453 g of TEA were added to 7.500 g of aqueous solution of thermosetting pluronic PL-F127 (25%). The mixture was homogenised by vigorous stirring, and while maintaining stirring the resulting solution was added with 0.1 mL of Eosin Y solution (10.9 mg/mL), 0.1 mL of fluorescein solution (10.9 mg/mL) and 0.1 mL of NVP solution. Then, the resulting mixture was casted in petri dishes and illuminated by the blue light of Example 1 for 30 seconds to form poly(hydroxyethyl acrylamide)/PL-F127-B cross-linked with PEGDA. The volume casted was controlled such as the thickness of the formed hydrogel was around 2 mm.

Light emitted through the polymer and fluorescence emitted by the polymer was measured using a SP-100 spectroradiometer (SP-100, ORB Optronix).

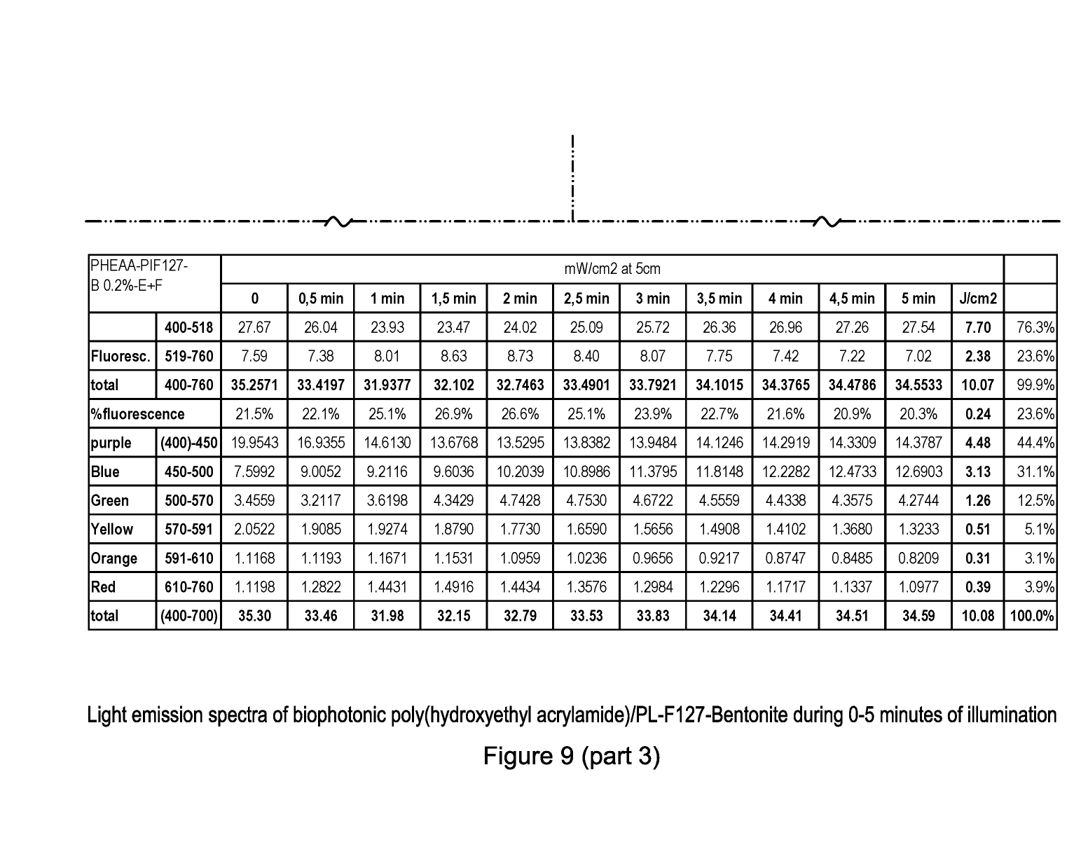

FIG. 9 shows the light spectrum recorded during exposure of the biophotonic membrane to blue light for 5 minutes. As can be seen, the emitted fluorescence in this case was not lower than that observed in Example 4 (the membranes contained the same concentration of Eosin Y and fluorescein), however, comparison to the emitted fluorescence exhibited by the membrane observed in Example 4, the membrane in this Example 6 exhibited a decreased amount of purple and blue light being emitted, and with respect to the green, yellow, orange and red light emitted there was an increase in the amount of each of these three colors emitted from the biophotonic hydrogel of Example 6.

### Example 7: Hydrogel of poly(hydroxyethyl acrylamide)/PL-F127-SiO₂

Amounts of 2.012 g of HEAA, 0.528 g of PEGDA, 0.150 g of fumed silica (SiO₂) and 0.0453 g of TEA were added to 7.500 g of aqueous solution of thermosetting pluronic PL-F127 (25%). The mixture was homogenised by vigorous stirring, and while maintaining stirring the resulting solution was added with 0.1 mL of Eosin Y solution (10.9 mg/mL), 0.1 mL of fluorescein solution (10.9 mg/mL) and 0.1 mL of NVP solution. Then, the resulting mixture was casted in petri dishes and illuminated by the blue light of Example 1 for 30 seconds to form poly(hydroxyethyl acrylamide)/PL-F127-SiO₂ cross-linked with PEGDA. The volume casted was controlled such as the thickness of the formed hydrogel was around 2 mm.

Light emitted through the polymer and fluorescence emitted by the polymer was measured using a SP-100 spectroradiometer (SP-100, ORB Optronix).

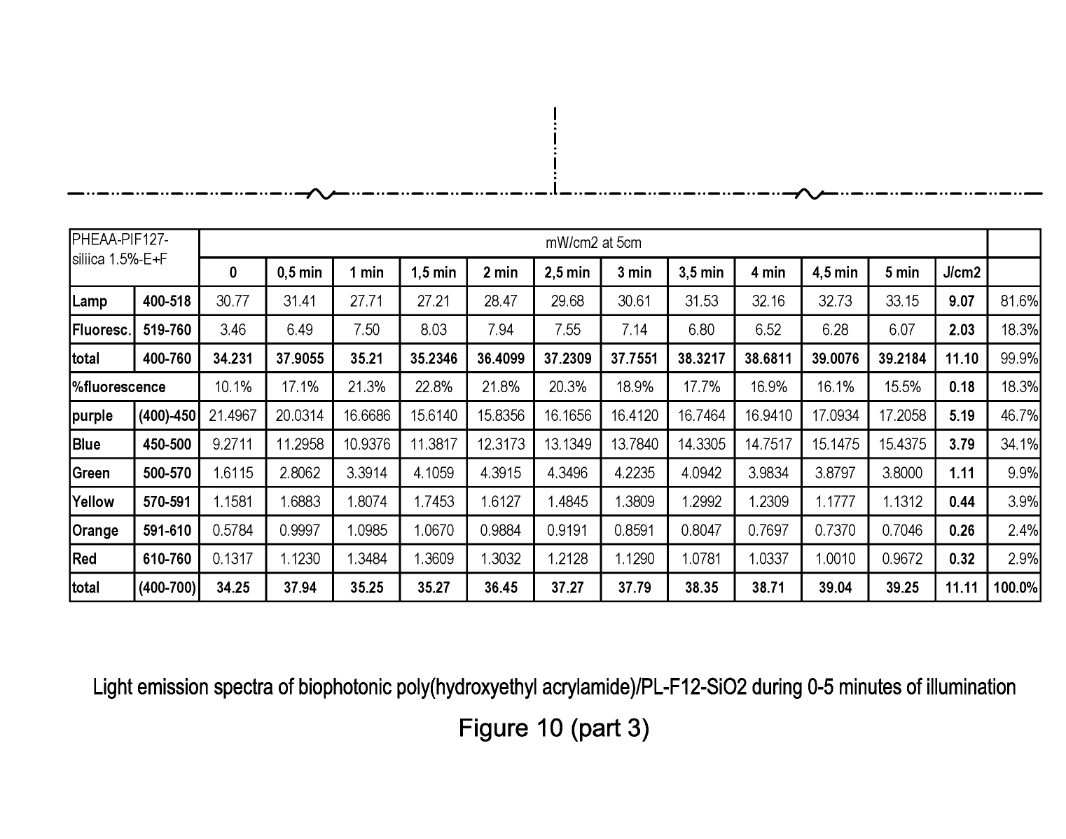

FIG. 10 shows the light spectrum recorded during exposure of the biophotonic membrane to blue light for 5 minutes. As can be seen, the emitted fluorescence in this case was not lower than that observed in Example 4 (the membranes contained the same concentration of Eosin Y and fluorescein), however, comparison to the emitted fluorescence exhibited by the membrane observed in Example 4, the membrane in this Example 6 exhibited a decreased amount of purple and blue light being emitted, and with respect to the yellow, orange and red light emitted there was a slight increase in the amount of each of these three colors emitted from the biophotonic hydrogel of Example 6.

### Example 8: Hydrogel of poly(hydroxyethyl acrylamide)/PL-F127-SiO₂—CTAB

Amounts of 2.068 g of HEAA, 0.501 g of PEGDA, 0.081 g of CTAB, 0.151 g of fumed silica (SiO2) and 0.0453 g of TEA were added to 7.500 g of aqueous solution of thermosetting pluronic PL-F127 (25%). The mixture was homogenised by vigorous stirring, and while maintaining stirring the resulting solution was added with 0.1 mL of Eosin Y solution (10.9 mg/mL), 0.1 mL of fluorescein solution (10.9 mg/mL) and 0.1 mL of NVP solution. Then, the resulting mixture was casted in petri dishes and illuminated by the blue light of Example 1 for 30 seconds to form poly(hydroxyethyl acrylamide)/PL-F127-SiO₂-CTAB cross-linked with PEGDA. The volume casted was controlled such as the thickness of the formed hydrogel was around 2 mm.

Light emitted through the polymer and fluorescence emitted by the polymer was measured using a SP-100 spectroradiometer (SP-100, ORB Optronix).

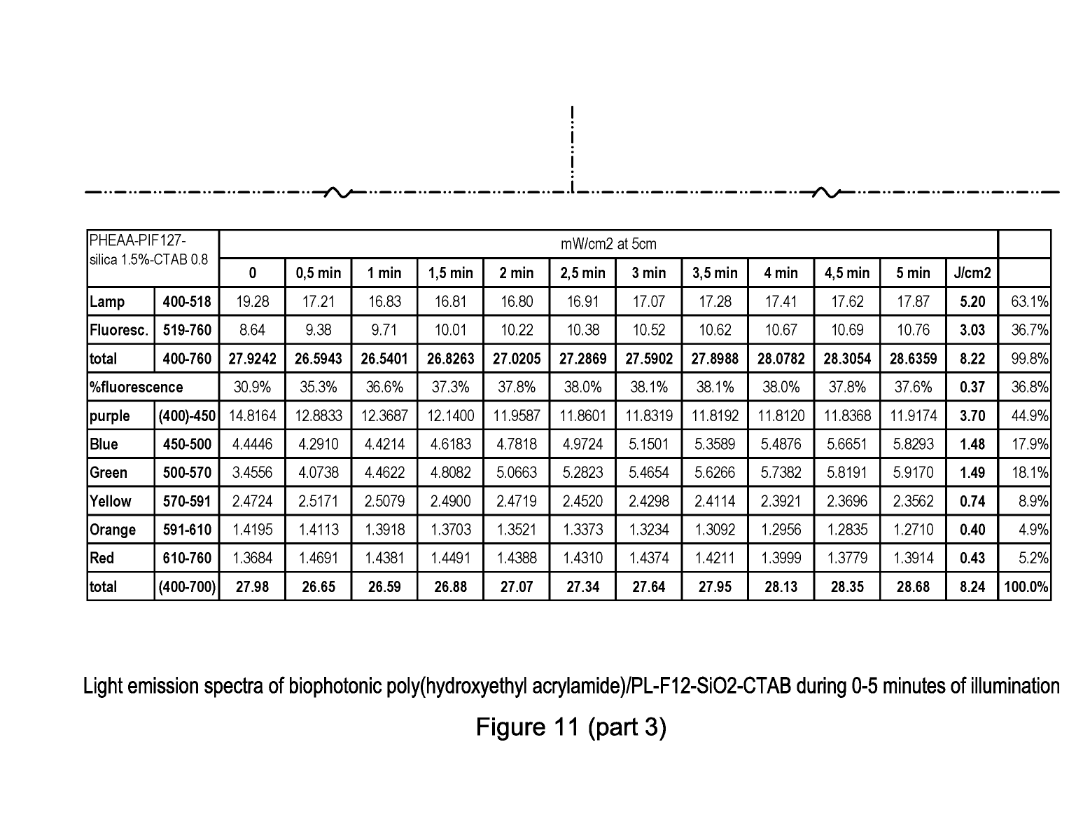

FIG. 11 shows the light spectrum recorded during exposure of the biophotonic membrane to blue light for 5 minutes. As can be seen, the emitted fluorescence in this case was less than that emitted by the biophotonic hydrogel of Example 5, but more than that emitted by the biophotonic hydrogel of Example 4. (the membranes contained the same concentration of Eosin Y and fluorescein). In comparison to the emitted fluorescence exhibited by the membrane observed in Example 4, the membrane in this Example 7 exhibited a significantly decreased amount of purple and blue light being emitted, and with respect to the green, yellow, orange and red light emitted there was a significant increase in the amount of each of these four colors emitted from the biophotonic hydrogel of Example 7, however, in comparison to the biophotonic membrane of Example 5, there was a lesser amount of these four colors emitted from the biophotonic membrane.

### Example 9: Modulation of IL6 and IL8 in HaCaT Cells by Biophotonic Polymer Membranes of Examples 1-4

The biophotonic hydrogels of Examples 1-4 were evaluated for their ability to modulate inflammation, specifically cytokines IL6 and IL8. HaCaT human keratinocyte cells were used as an accepted in vitro model for assessing modulation of these inflammatory cytokines.

Excessive, uncontrolled inflammation is observed in many skin conditions as well as in wounds, and can be detrimental to a host such as by impairing wound healing processes. Therefore a down regulation of IL6 and IL8 secretion may be beneficial in wound healing as well as alleviating other conditions, such as eczema and psoriasis.

A non-toxic concentration of IFNγ was used to modulate the secretion of IL6 and IL8 by the HaCaT cells. Dexamethasone (final concentration of 5 uM) was used as a positive control (strong inhibitor of pro-inflammatory cytokine production). The potential toxic effect of light on HaCaT cells was assessed using an in vitro toxicology assay kit, XTT based, which is a spectrophotometric evaluation of viable cell number.

Cells cultures were illuminated with light emitted by and transmitted through the polymer membranes of Examples 1-4. The membranes were positioned 5 cm above the cell cultures and the membranes were illuminated with blue light having a peak wavelength between 400-470 nm and a power density of about 30-150 mW/cm² for 90 seconds.

Cytokine quantification was performed by cytokine ELISA on the culture supernatant 24 hours after illumination according to manufacturer instructions (DuoSet ELISA development kit from R&D Systems). The quantity of cytokine secreted was normalized to cell viability. No toxic effect was observed for all the test samples as measured by cell viability using a spectrophotometric evaluation of viable cell number 24 hours after treatment. All samples were screened in quadruplets. Three repetitions were performed for each of the tested membranes.

It was found that the light emitted by the eosin and fluorescein from the biophotonic hydrogels of Examples 1-4 produced a downward modulation of IL6 and IL8 on the IFNγ stimulated HaCaT cells.

Table 1 summarizes the light treatment being received by the cultured cells during the illumination time from each polymer. Table 2 summarizes the IL6 and IL8 expression after illumination with each of the polymers.

TABLE 1
Light treatment being received by the cultured HaCaT cells during the illumination
time from each membrane

| | | J/cm2, for 90 sec exposure, THERA lamp at 5 cm | | | | | |
|---|---|---|---|---|---|---|---|
| | | Violet | Blue | Green | Yellow | Orange | Red |
| Membrane 1 | PHEAA eosin/fluorescein | 3.57 | 1.28 | 0.3 | 0.07 | 0.04 | 0.04 |
| | (0.011% each) | | | | | | |
| Membrane 2 | PHEAA/gelatine eosin/fluorescein | 0.84 | 0.29 | 0.21 | 0.08 | 0.05 | 0.08 |
| | (0.011% each) | | | | | | |
| Membrane 3 | PHEA/HEC eosin/fluorescein | 2.96 | 0.81 | 0.18 | 0.08 | 0.03 | 0.02 |
| | (0.011% each) | | | | | | |
| Membrane 4 | PHEAA/PL-F127 eosin/fluorescein | 2.21 | 1.29 | 0.35 | 0.15 | 0.09 | 0.1 |
| | (0.011% each) | | | | | | |

TABLE 2
IL6 and IL8 expression after illumination from each membrane.

| | Decrease of IL6 in | Decrease of IL8 in |
|---|---|---|
| | IFNgamma activated | IFNgamma activated |
| | HaCaT cells | HaCaT cells |
| Membrane 1 | +++ | + |
| Membrane 2 | ++ | + |
| Membrane 3 | +++ | + |
| Membrane 4 | ++ | + |

### CONCLUSIONS

The results of the experiments revealed that matrices which allow blue light penetration (up to 5 J/cm² of energy fluence delivered to the cells) are the most effective in pro-inflammatory cytokines IL-6 downregulation. 62% and 57% decrease in IL-6 production was observed for PHEAA and PHEAA/HEC matrices, respectively;

Matrices which generate the highest fluorescence within green and red light spectrum are the most effective in modulating IL-8 secretion. 24% and 28% reduction in IL-8 production was observed for PHEAA and PHEAA/PL-F127 matrices, respectively. Interestingly the same matrices are potent at downmodulating IL-6 secretion, suggesting that the combination of blue, green and red fluorescence is required to achieve the optimal therapeutic effect;

Possibly the generation of matrices with the ability to generate higher fluorescence within red light spectrum would enhance the downmodulatory effect on pro-inflammatory cytokines during inflammatory phase of wound healing process.

### Example 10: Modulation of Collagen Production by Biophotonic Polymer Membranes of Examples 1-4

Human Dermal Fibroblasts (DHF) cells were used as an in vitro model to study the effect of visible blue light in combination with embodiments of the biophotonic polymer membranes of the present disclosure on the secretion of one of the extracellular matrix (ECM) components, collagen.

Collagen production may be useful in wound healing, as well as other indications such as skin conditions and rejuvenation. In wound healing, within four-five days upon injury, matrix-generating cells (i.e. fibroblasts), move into the granulation tissue. These fibroblasts degrade the provisional matrix via matrix metalloproteinases (MMPs) and respond to cytokine/growth factors by proliferating and synthesizing new extracellular matrix (ECM) which is composed of collagen I, III, and V, proteoglycans, fibronectin and other components. TGF-beta concurrently inhibits proteases while enhancing protease inhibitors, favoring matrix accumulation.

A non-toxic concentration of TGFβ-1 was added to the cells to mimic hyperproliferation conditions. The potential toxic effect of light on the cells was assessed using an in vitro toxicology assay kit, XTT based, which is a spectrophotometric evaluation of viable cell number.

Cell cultures were illuminated with light emitted by and transmitted through the polymer membranes of Examples 1-4. The membranes were positioned 5 cm above the cell cultures and the membranes were illuminated with blue light having a peak wavelength between 400-470 nm and a power density of about 30-150 mW/cm² for 5 minutes. Vitamin C and TGFβ-1 was used as a positive control.

Forty eight hours after treatment, collagen production was evaluated using the Picro-Sirius red method. In brief, collagen molecules being rich in basic amino acids strongly react with acidic dyes. Sirius red is an elongated dye molecule which reacts with collagen (type I, II, V), binds to it, and after several washes which remove free dye, the bound Sirius red is eluted with sodium hydroxide and quantified using a spectrophotometer. All samples were screened in quadruplets. Two repetitions were performed for each of the tested matrices.

Table 3 summarizes the different lights and the radiant fluencies received by the cultured cells during the illumination time from each polymer.

TABLE 3
Light treatment being received by the cultured DHF during the illumination time from each

| | | J/cm2, for 5 min exposure, THERA lamp at 5 cm | | | | | |
|---|---|---|---|---|---|---|---|
| | | Violet | Blue | Green | Yellow | Orange | Red |
| Membrane 1 | PHEAA eosin/fluorescein | 13.48 | 6.83 | 0.91 | 0.21 | 0.12 | 0.13 |
| | (0.011% each) | | | | | | |
| Membrane 2 | PHEAA/gelatine eosin/fluorescein | 3.28 | 1.29 | 0.67 | 0.26 | 0.16 | 0.25 |
| | (0.011% each) | | | | | | |
| Membrane 3 | PHEAA/HEC eosin/fluorescein | 10.57 | 5.14 | 0.85 | 0.36 | 0.21 | 0.22 |
| | (0.011% each) | | | | | | |
| Membrane 4 | PHEAA/PL-F127 eosin/fluorescein | 7.32 | 5.07 | 1.1 | 0.37 | 0.22 | 0.28 |
| | (0.011% each) | | | | | | |

Table 4 shows the collagen production in TGF-beta1 stimulated DHF cells after illumination with each of the polymers of Examples 1-4.

TABLE 4
Collagen production in TGFβ1-stimulated
DHF cells after illumination from each membrane

| | Collagen concentration increase in DHF |
|---|---|
| | cultured supernatant after illumination |
| | through biophotonic membrane |
| Membrane 1 | +++ |
| Membrane 2 | ++ |
| Membrane 3 | ++ |
| Membrane 4 | +++ |

### CONCLUSIONS

The results of the Picro-Sirius red assay showed that matrices which generate the highest fluorescence within red light spectrum (up to 0.28 J/cm² of energy fluence delivered to the cells) are the most effective in stimulating collagen production. 5-, 6,3-, and 6,5-fold increase in collagen production in DHF cell culture supernatant was observed upon illumination with PHEAA/Gelatin; PHEAA/HEC, and PHEAA/PL-F127 matrices, respectively.

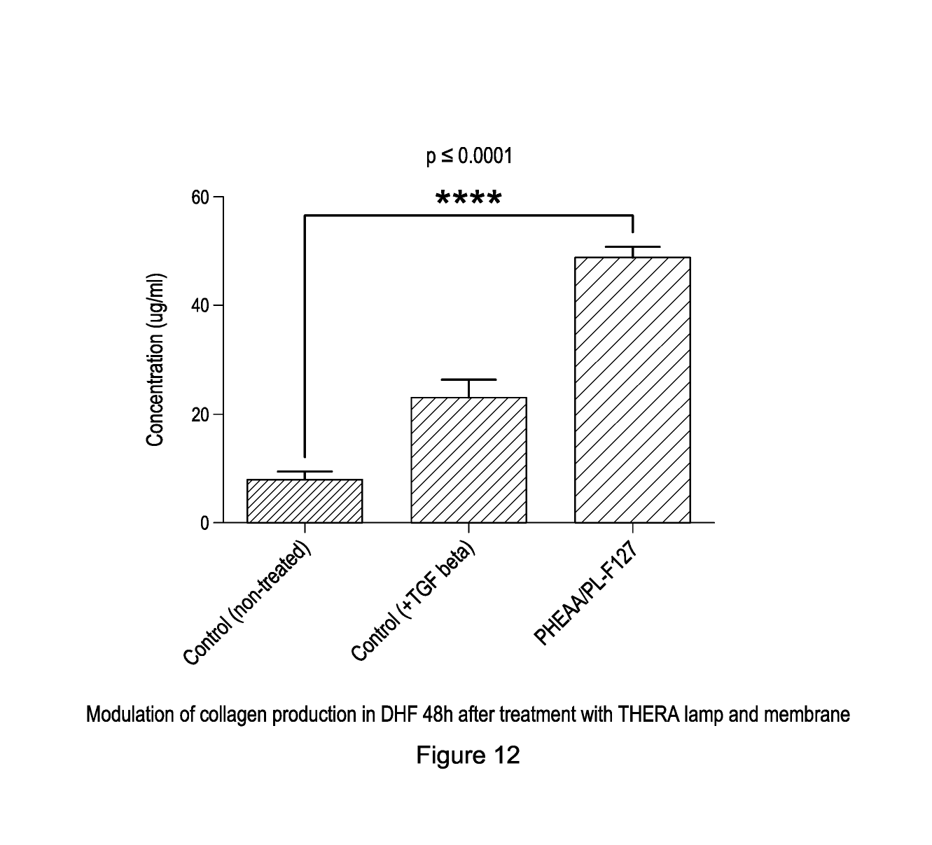

Interestingly the same matrices i.e. PHEAA/Gelatin; PHEAA/HEC, and PHEAA/PL-F 127 generate high fluorescence within green light spectrum, suggesting that deeper penetrating light such as green and red modulate together collagen synthesis in DHF (FIG. 12).

### Example 11: Cytokines and Growth Factors in DHF

In order to gain more detailed picture of the biological effect mediated by tested matrices, Human Cytokine Antibody Array (RayBio C-Series, RayBiotech, Inc.) was performed. Cytokines broadly defined as secreted cell-cell signaling proteins play important roles in inflammation, innate immunity, apoptosis, angiogenesis, cell growth and differentiation. Simultaneous detection of multiple cytokines provides a powerful tool to study cell activity. Regulation of cellular processes by cytokines is a complex, dynamic process, often involving multiple proteins. Positive and negative feedback loops, pleiotropic effects and redundant functions, spatial and temporal expression of or synergistic interactions between multiple cytokines, even regulation via release of soluble forms of membrane-bound receptors, are all common mechanisms modulating the effects of cytokine signaling.

The effect of light/biophotonic membranes on cytokine secretion profile in the culture medium by DHF and THP-1 (Example 12 below) cells (prior to light illumination the THP-1 cells were differentiated in macrophages by adding phorbol 12-myristate 13-acetate (PMA)) was determined using Human Cytokine Antibody Array (RayBio C-series from Raybiotech). In brief, a non-toxic concentration of TGF β-1 was used to stimulate DHF cells. In case of differentiated THP-1 cells into macrophages (Example 8 below), IFNγ and LPS were used to stimulate cells into an inflammatory phenotype. DHF and THP-1 cells supernatants were collected 24 h post-illumination and incubated with arrayed antibody membranes according to the manufacturer instructions. Obtained signals were quantified with ImageJ (U.S. National Institute of Health) software. For each experiment, the XTT assay was performed to normalize the quantity of cytokine secreted to the cell viability (in all cases the viability was over 90% showing a non-toxic effect of the treatment). All samples were done in quadruplets.

The effect of illuminated membrane on cytokines and growth factor secretion in DHF and THP-1 (Example 12 below) cells is summarized in the Tables 5 and 6, respectively below.

TABLE 5
Modulation of protein expression in DHF activated
by TGFβ1 24 hours after treatment with THERA lamp
and matrices compared to control untreated cells.

| | Membrane 1 | Membrane 4 |
|---|---|---|
| Cytokines | | |
| IL2 | ↑ | ↑ |
| IL3 | ↓↓↓ | ↓↓↓ |
| IL4 | ↑ | ↑ |
| IL6 | ↓ | ↓↓↓ |
| IL8 | ↓↓↓ | ↓↓↓ |
| IL10 | ↑↑↑ | ↑↑↑ |
| IL12 p40/70 | — | — |
| IL13 | ↓ | ↓ |
| IL15 | ↑ | ↑ |
| TNFalpha | ↓ | ↓ |
| TNFbeta | ↓↓↓ | ↓↓ |
| IL1alpha | ↓↓↓ | ↓↓↓ |
| IL1beta | ↓ | ↓↓↓ |
| IFNgamma | ↓↓↓ | ↓↓↓ |
| MCP1 | ↓↓↓ | ↓↓↓ |
| MCP2 | ↓ | ↓ |
| MCP3 | ↑ | ↑ |
| M-CSF | ↓↓↓ | ↓↓↓ |
| MDC | | ↑↑↑ |
| MIG | ↑↑↑ | ↑↑↑ |
| MIP-1belta | ↓↓↓ | ↓↓↓ |
| RANTES | ↓↓↓ | ↓↓↓ |
| TARC | ↑ | ↑ |
| Growth Factors | | |
| EGF | — | — |
| IGF-1 | ↑↑↑ | ↑↑↑ |
| ANG | ↑ | ↑↑↑ |
| VEGF | ↑↑ | ↑↑↑ |
| PDGF-BB | ↓↓↓ | ↓↓↓ |
| ENA-78 | ↓↓↓ | ↓↓ |
| G-CSF | ↑↑↑ | ↑↑↑ |
| GM-CSF | ↓↓↓ | ↓↓↓ |
| GRO | ↓↓↓ | ↓↓↓ |
| GROalpha | ↓↓↓ | ↓↓ |
| TGFbeta1 | ↓↓↓ | ↓↓↓ |
| Leptin | — | — |

↓ less than 25% decrease
↓↓ 25-50% decrease
↓↓↓ more than 50% decrease
— No modulation
↑ less than 25% increase
↑↑ 25-50% increase
↑↑↑ more than 50% increase

### Example 12: Cytokines and Growth Factors in Macrophages

The methodology of Example 11 was carried out on macrophages which were illuminated using the method of Example 11 and using membrane 1.

TABLE 6
Modulation of protein expression in THP1 cells (differentiated
into macrophages) 24 hours after treatment with Thera ™ lamp
and matrices compared to control untreated cells.

| | Membrane 1 |
|---|---|
| Cytokines | |
| IL2 | — |
| IL3 | ↑↑↑ |
| IL4 | — |
| IL6 | ↓↓↓ |
| IL8 | ↓ |
| IL10 | ↑↑↑ |
| IL12 p40/70 | ↑ |
| IL13 | — |
| IL15 | ↑ |
| TNFlpha | ↓↓↓ |
| TNFbeta | — |
| IL1alpha | ↓ |
| IL1beta | ↓↓ |
| IFNgamma | ↓↓↓ |
| MCP1 | ↓↓ |
| MCP2 | — |
| MCP3 | — |
| M-CSF | ↑ |
| MDC | ↑ |
| MIG | — |
| MIP-1 delta | ↑ |
| RANTES | ↓↓ |
| TARC | ↑ |
| Growth Factors | |
| EGF | — |
| IGF-1 | — |
| ANG | — |
| VEGF | ↑ |
| PDGF-BB | ↑ |
| ENA-78 | — |
| G-CSF | — |
| GM-CSF | ↑↑↑ |
| GRO | — |
| GROalpha | ↑↑↑ |
| TGFbeta1 | ↑↑↑ |
| Leptin | ↑ |

↓ less than 25% decrease
↓↓ 25-50% decrease
↓↓↓ more than 50% decrease
— No modulation
↑ less than 25% increase
↑↑ 25-50% increase
↑↑↑ more than 50% increase

The results of the experiments revealed that the biophotonic membranes 1 and 4 are effective in pro-inflammatory cytokines (such as IL6, IL8, TNF alpha, IL1 beta and IFN gamma) downregulation in DHF and THP1 macrophages cells, respectively.

PHEAA and PHEAA/PL-F127 polymers proved to be efficient at down modulation of cytokines (such as MCP1 and RANTES) involved in inflammatory conditions.

### Example 13: Proliferation Level in DHF Cells Upon Biophotonic Membrane Illumination

Fibroblast migration to and proliferation within the wound site are prerequisites for wound granulation and healing. Fibroblasts then participate in the construction of scar tissue and its remodeling. Thus viable, actively dividing fibroblasts are a crucial player in healing progression.

The XTT based method measures the mitochondrial dehydrogenase activity of proliferating cells. In brief, the mitochondrial dehydrogenases of viable cells reduce the tetrazolium ring of XTT, yielding an orange derivative, which is water soluble. The absorbance of the resulting orange solution is measured spectrophotometrically. An increase or decrease in cells number relative to control cells, results in an accompanying change in the amount of orange derivative, indicating the changes in the number of viable, dividing cells.

DHF cells were illuminated for 5 min with biophotonic membrane 4 (PHEAA/PL-F127) and 24 h post-treatment an XTT solution was added to the cells. Four hours later the absorbance of orange supernatant was measured spectrophotometrically. The difference in the number of actively proliferating fibroblasts as compared to non-illuminated control were calculated.

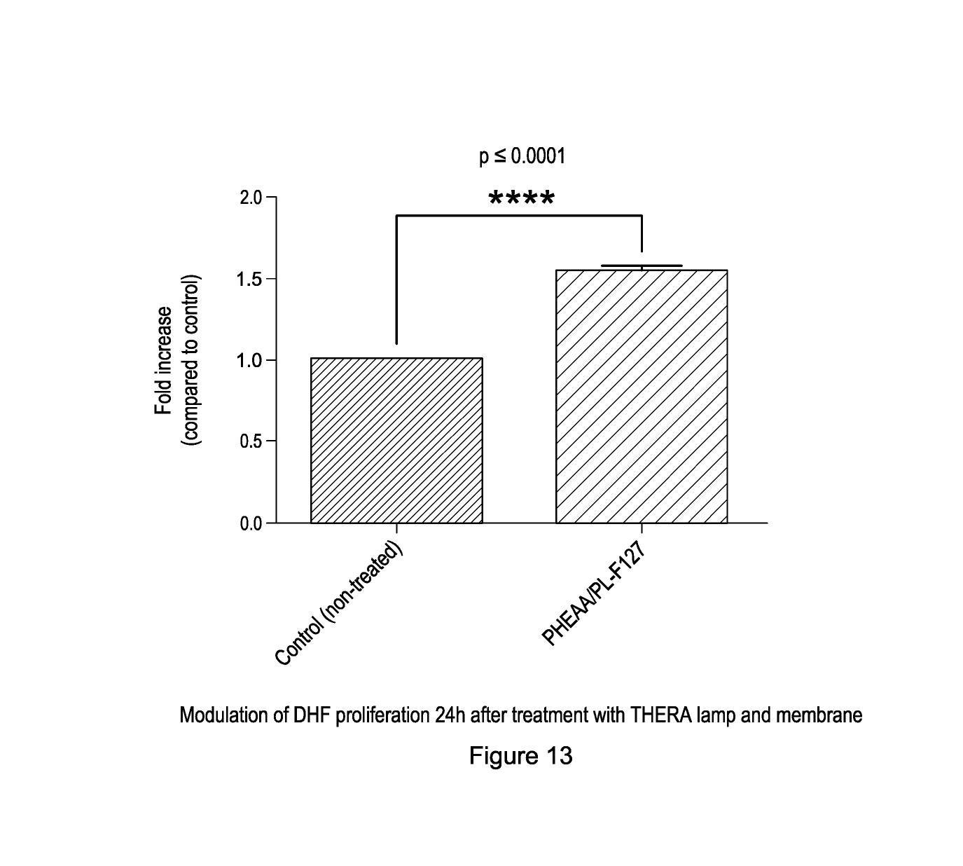

The data showed that the polymer membrane 4 induces proliferation of DHF compared to a control. In publications, proliferation of up to about 25-30% was seen. In the present case, an up to 50% proliferation was observed (FIG. 13).

### Example 14: Evaluation of Biological Properties of Biophotonic PHEAA/Pl-F127 Formulation in a Wound Healing Process

Injury to the skin initiates a cascade of events that overlap in time and space, including inflammation, new tissue formation, and tissue remodeling, which finally lead to at least partial reconstruction of the wounded area. The repair process is initiated immediately after injury by release of various cytokines, growth factors, and low-molecular-weight compounds. During the early inflammation step, cells debris and bacteria are eliminated by the presence of phagocytic cells such as leukocytes and macrophage M1. Later inflammation response is essential for generating growth factor and cytokines signals that induce cell migration, proliferation, differentiation and ECM component synthesis necessary for tissue repair (Eming S A, Krieg T, Davidson J M. Inflammation in wound repair: molecular and cellular mechanisms. J Invest Dermatol, 2007; 127:514-525). Excessive inflammation activity may have a profound impact on the quality of the healing. Chronic wounds are characterized by persistent inflammation, disturbed pattern of growth factors production and excessive proteinase activity of MMPs (Eming et al., 2007; Loots M A, Lamme E N, Zeegelaar J, Mekkes J R, Bos J D, Middelkoop E. Differences in cellular infiltrate and extracellular matrix of chronic diabetic and venous ulcers versus acute wounds. J Invest Dermatol. 1998; 111:850-857, Schultz G S, Mast B A. Molecular analysis of the environments of healing and chronic wounds: cytokines, proteases and growth factors. Wounds, 1998; 10(suppl. F):1F-9F). A marked inflammation and disturbed growth factors production and enzymes activity is also found in other skin diseases such as atopic dermatitis and psoriasis.

The excessive, uncontrolled inflammation is detrimental to the host and negatively influence granulation, reepithalisation and scar formation process; therefore the purpose of this study was to evaluate the ability of the PHEAA/PLF127 hydrogel, on illumination with a KLOX Multi-LED light at 5 cm distance, to control and decrease the inflammation, thus promoting next phases of the wound healing. Without being bound to any particular hypothesis, this phenomenon could be achieved by induction of wide variety of different type of mediators, growth factors and enzymes accelerating resolution of the inflammation and promoting wound contraction and re-epithalisation.

The PHEAA/PLF127 hydrogel, which is a liquid formulation, is a vehicle that may be used with a broad variety of chromophores and such a chromophore-containing formulation is photo-polymerized within about 30 seconds upon being illuminated with a blue light (such as with a KLOX Multi-LED light) placed at 5 cm distance. The PHEAA/PLF127 hydrogel can be applied in a liquid form and thereafter illuminated in order to photo-polymerized, after which the biophotonic treatment follows immediately. Alternatively, the PHEAA/PLF127 hydrogel chromophore-containing formulation may be photo-polymerized for 30 sec before application, and this latter procedure was used in all experiments described in this Example 14. After treatment, the polymerized PHEAA/PLF127 hydrogel is easily removed as the polymerized polymer is pealable. During the polymerization process, the formulation does not release any significant amount of heat, and post-treatment, the polymerized formulation may feel cool on the skin of a treated subject.

In the experiments presented below in this Example 14, the PHEAA/PLF127 hydrogel contained two chromophores, eosin Y and fluorescein, in equal percentage-in-weight amount as between the two chromophores (109 micrograms per 1 gram of hydrogel for each chromophore). The pre-formed polymerized formulation may be sterilized by autoclave, or in its liquid form by filtration using a 0.22 um filter without any change in both the polymerization capacity

### Experimental Design

a) Protein Secretion

Dermal Human Fibroblasts (DHF) and a 3D skin model were used as in vitro models to study the effect of PHEAA/PL-F127 in combination with blue light on the secretion of inflammatory mediators, growth factors, tissue remodeling proteins (such as matrix metalloproteinases (MMPs), and tissue inhibitors of matrix metalloproteinases (TIMPs). The cells were illuminated with different power densities using PHEAA/PL-F127 and visible blue light (KLOX Multi-LED light) at the distance of 5 cm. Blue light and fluorescence dose received by the cells during the illumination time are shown in Table 7.

DHF were cultured on glass bottom dish. One hour prior to illumination cells were treated with non-toxic concentration of IFN-γ (300 U/ml) to induce the inflammatory state observed in acute and chronic wounds. IFN-γ was maintained in the culture medium after the illumination to mimic the inflammatory condition through whole time during which the assay was performed. PHEAA/PL-F127 was applied on the other side of the glass dish and illuminated at 5 cm distance using blue visible light (KLOX Thera™ lamp). Increasing radiant fluencies (J/cm²) were used to illuminate DHF. Cells were also treated with light alone, which served as an internal control to ensure whether the combination of light with the PHEAA/PL-F127 exerted a significant biological effect compared to light alone. At 24 h, 48 h, and 72 h post-treatment, supernatant was collected and arrays were performed to evaluate the inflammatory cytokines, chemokines, growth factors, MMPs and TIMPs production profile upon PHEAA/PL-F127 treatment. The lists of proteins analyzed for each array are described below in Tables 8, 9 and 10:

Antibodies Array Profiles

TABLE 8
Human Cytokine Antibody Array C3

| | A | B | C | D | E | F | G | H | I | J | K | L |
|---|---|---|---|---|---|---|---|---|---|---|---|---|
| 1 | POS | POS | NEG | NEG | ENA-78 | G-CSF | GM-CSF | GRO | GRO | I-309 | IL-1 | IL-1 |
| | | | | | | | | | alpha | | alpha | beta |
| 2 | POS | POS | NEG | NEG | ENA-78 | G-CSF | GM-CSF | GRO | GRO | I-309 | IL-1 | IL-1 |
| | | | | | | | | | alpha | | alpha | beta |
| 3 | IL-2 | IL-3 | IL-4 | IL-5 | IL-6 | IL-7 | IL-8 | IL-10 | IL-12 | IL-13 | IL-15 | IFN |
| | | | | | | | | | P40/70 | | | gamma |
| 4 | IL-2 | IL-3 | IL-4 | IL-5 | IL-6 | IL-7 | IL-8 | IL-10 | IL-12 | IL-13 | IL-15 | IFN |
| | | | | | | | | | P40/70 | | | gamma |
| 5 | MCP-1 | MCP-2 | MCP-3 | M-CSF | MDC | MIG | MIP-1 | RANTES | SCF | SDF-1 | TARC | TGF |
| | | | | | | | delta | | | | | beta 1 |
| 6 | MCP-1 | MCP-2 | MCP-3 | M-CSF | MDC | MIG | MIP-1 | RANTES | SCF | SDF-1 | TARC | TGF |
| | | | | | | | delta | | | | | beta 1 |
| 7 | TNF | TNF | EGF | IGF-1 | ANG | OSM | THPO | VEGF | PDGF | Leptin | NEG | POS |
| | alpha | beta | | | | | | | BB | | | |
| 8 | TNF | TNF | EGF | IGF-1 | ANG | OSM | THPO | VEGF | PDGF | Leptin | NEG | POS |
| | alpha | beta | | | | | | | BB | | | |

POS = Positive Control Spot
NEG = Negative Control Spot
BLANK = Blank Spot

TABLE 9
Human Growth Factor Antibody Array C1

| | A | B | C | D | E | F | G | H | I | J | K | L |
|---|---|---|---|---|---|---|---|---|---|---|---|---|
| 1 | POS | POS | NEG | NEG | AREG | bFGF | b-NGF | EGF | EGFR | FGF-4 | FGF-6 | FGF-7 |
| 2 | POS | POS | NEG | NEG | AREG | bFGF | b-NGF | EGF | EGFR | FGF-4 | FGF-6 | FGF-7 |
| 3 | G-CSF | GDNF | GM | HB | HGF | IGFBP | IGFBP | IGFBP | IGFBP | IGFBP | IGF-1 | IGF-1 |
| | | | CSF | EGF | | 1 | 2 | 3 | 4 | 6 | | sR |
| 4 | G-CSF | GDNF | GM | HB | HGF | IGFBP | IGFBP | IGFBP | IGFBP | IGFBP | IGF-1 | IGF-1 |
| | | | CSF | EGF | | 1 | 2 | 3 | 4 | 6 | | sR |
| 5 | IGF-2 | M-CSF | M-CSF | NT-3 | NT-4 | PDGF R | PDGF R | PDGF | PDGF | PDGF | PLGF | SCF |
| | | | R | | | alpha | beta | AA | AB | BB | | |
| 6 | IGF-2 | M-CSF | M-CSF | NT-3 | NT-4 | PDGF R | PDGF R | PDGF | PDGF | PDGF | PLGF | SCF |
| | | | R | | | alpha | beta | M | AB | BB | | |
| 7 | SCF | TGF | TGF | TGF | TGF | VEGF | VEGF | VEGF | VEGF | BLANK | BLANK | POS |
| | R | alpha | beta | beta 2 | beta 3 | | R2 | R3 | D | | | |
| 8 | SCF | TGF | TGF | TGF | TGF | VEGF | VEGF | VEGF | VEGF | BLANK | BLANK | POS |
| | R | alpha | beta | beta 2 | beta 3 | | R2 | R3 | D | | | |

POS = Positive Control Spot
NEG = Negative Control Spot
BLANK = Blank Spot

TABLE 10
Human Matrix Metalloproteinase Antibody Array C1.

| | A | B | C | D | E | F | G | H |
|---|---|---|---|---|---|---|---|---|
| 1 | POS | POS | NEG | NEG | MMP-1 | MMP-2 | MMP-3 | MMP-8 |
| 2 | POS | POS | NEG | NEG | MMP-1 | MMP-2 | MMP-3 | MMP-8 |
| 3 | MMP-9 | MMP-10 | MMP-13 | TIMP-1 | TIMP-2 | TIMP-4 | NEG | POS |
| 4 | MMP-9 | MMP-10 | MMP-13 | TIMP-1 | TIMP-2 | TIMP-4 | NEG | POS |

POS = Positive Control Spot
NEG = Negative Control Spot
BLANK = Blank Spot

For the 3D skin model experiment, EpiDerm full thickness tissue (also referred to as 3D skin) that consists of Normal Human-derived Epidermal Keratinocytes (NHEK) and Dermal Fibroblasts (NHFB) was used. A wound was created inside the insert before treatment, and at 24 hours post-treatment, supernatants were collected for proteins arrays as described above. The polymerized membrane was placed above a nylon mesh itself layered on the surface of the 3D skin insert. The nylon mesh contains two notches for easy removal of the polymerized membrane after treatment because the 3D skin inserts were placed deep in the dish. The nylon mesh does not interfere with the radiant fluencies delivered to the samples.

To assess the potential cytotoxicity of the treatment, supernatants from the treated cell cultures and 3D skin inserts were also screened for lactate dehydrogenase (LDH) activity. LDH is an intracellular enzyme that is released in the culture medium when the cell is damaged. It is a marker of cytotoxicity. The assay quantifies the LDH activity that reduces NAD to NADH. NADH is specifically detected by colorimetry.

b) Cell Proliferation

Prior to the treatment cells were undergoing starvation (medium deprived of serum and hormones) in order to be synchronised in G1 phase. Cells were monitored for the proliferation at 24 h, 48 h, and 72 h post-treatment using CyQUANT direct cell proliferation assay.

c) Total Collagen Production

DHF cells were cultured to achieve logarithmic growth phase and subsequently illuminated with PHEAA/PL-F127 and visible blue light (KLOX Multi LED light) at power density of 14.4 J/cm², at 5 cm distance. TGF-β1 and vitamin C were used as a positive control for the experiment purposes. At 48 h post-illumination supernatants were collected and screened for total collagen content using SIRCOL total collagen assay.

Results

a)(i) PHEAA/PL-F127-Mediated Effect on Inflammatory Mediators Production in Dermal Human Fibroblast

At 24 h, 48 h, and 72 h post-treatment supernatant was collected and inflammatory cytokine array was performed to evaluate the inflammatory cytokines production profile upon PHEAA/PL-F127 treatment in combination with KLOX Multi-LED light. The results of the array are summarized in Table 11.

Analysis of LDH activity showed that no significant cytotoxic effect of the treatment was observed in all PHEAA/PL-F127 illuminated samples.

TABLE 11
Summary of significant up (↑) and down-regulation (↓) observed
in inflammatory mediators production (cytokines in red, chemokines
in blue) compared to non-treated controls.

| | PHEAA/PLF127 with KLOX Multi LED light | | | |
|---|---|---|---|---|
| | 7 J/cm² | 14.4 J/cm² | 19.5 J/cm² | 24 J/cm² |
| | (2.5 min) | (5 min) | (7 min) | (10 min) |
| 24 h | ↑ IL3, IL8, | ↓IL4, IL6, | ↓IL3, IL4, | ↓IL4, IL6, |
| | IL10, TNFα, | TNFα, TNFβ, | IL6, TNFα, | IL8, TNFα, |
| | TNFβ, MCP2, | IL1α, IL1β; | TNFβ, IL1α, | TNFβ, IL1α, |
| | M-CSF, GRO, | ↑ IL8, IL10, | IL1β, MCP2, | IL1β, IFNγ, |
| | MIP1-Δ, | MCP2, GRO, | MCP3, M- | MCP2, MCP3, |
| | THPO; | THPO; | CSF, ENA78, | M-CSF, |
| | | | TARC, | ENA78, |
| | | | RANTES, | TARC, |
| | | | MIP1-Δ; | RANTES, |
| | | | ↑ IL8, IL10; | MIP1-Δ; |
| | | | | ↑MCP1 |
| 48 h | Not tested | Not tested | ↓IL4, IL5, | Not tested |
| | | | IL6, | |
| | | | IL12p40/70, | |
| | | | TNFα, TNFβ, | |
| | | | IL1α, IL1β, | |
| | | | IFNγ; | |
| | | | ↑IL2; | |
| 72 h | Not tested | Not tested | ↓IL4, IL5, | Not tested |
| | | | IL6, | |
| | | | IL12p40/70, | |
| | | | TNFα, TNFβ, | |
| | | | IL1α, IL1β, | |
| | | | IFNγ; | |
| | | | ↑IL2; | |

Results from the inflammatory cytokines array analysis indicated that biophotonic treatment utilizing the PHEAA/PL-F127 membrane mediated an anti-inflammatory effect, as observed in IFNγ stimulated human fibroblasts as a majority of tested pro-inflammatory cytokines and chemokines was significantly down-regulated following the biophotonic treatment.

From four different intensities tested, a power density of 19.5 J/cm² appeared to be a most effective at reducing production of pro-inflammatory cytokines (such as IL6, TNFα, TNFβ, IL1α, and IL1β) which are hallmarks of inflammation. Along with these cytokines, the level of other chemokines (such as MCP2, MCP3, M-CSF, ENA78, TARC, RANTES, and MIP1-Δ), which act as chemoattractant for the inflammatory cells to bring them to the site of inflammation, were also significantly reduced.

Under resting condition (no IFN-γ stimulation) no variation in the cytokine mediators level was observed upon the biophotonic PHEAA/PL-F127 treatment.

By controlling the duration and extent of an inflammation phase along with the level of major cytokine players, the biophotonic PHEAA/PL-F127 treatment may facilitate and accelerate the resolution of inflammation and allows the wound healing process to move to the next phases, such as granulation, re-epithelialization and remodeling.

a)(ii) PHEAA/PL-F127-Mediated Effect on Growth Factors Secretion in Dermal Human Fibroblast Culture

At 48 h, and 72 h post-treatment, supernatant was collected and a growth factor array was performed to evaluate the growth factors production profile upon the biophotonic PHEAA/PL-F127 treatment. The results of the array are summarized in Table 12.

TABLE 12
Summary of significant up - (↑) and down-regulation (↓) observed
in growth factors secretion compared to non-treated controls.

| | PHEAA/PL127 with KLOX Multi LED light | | |
|---|---|---|---|
| | 14.4 J/cm² | 19.5 J/cm² | 24 J/cm² |
| 48 h | No | ↑ IGFBP4, IGF2, M- | ↑ IGFBP6, IGF2, M- |
| | significant | CSF, G-CSF, GM-CSF, | CSF, M-CSFR, G-CSF, |
| | changes | Areg, bFGF, bNGF, HB, | GM-CSF, Areg, bFGF, |
| | observed | HGF, TGFβ, TGFβ2, | bNGF, EGF, FGF4, |
| | compare to | VEGF R3, PDGF AA, | HB; |
| | untreated | PDGF BB; | |
| | control | | |
| 72 h | No | ↑ IGFBP1, IGFBP2, | ↑ IGFBP1, IGFBP2, |
| | significant | IGFBP3, IGFBP4, IGF1, | IGFBP3, IGFBP4, |
| | changes | IGF1-sR, NT4, bFGF, | IGF2, M-CSF, M-CSF |
| | observed | FGF6, bNGF, EGF, | R, GM-CSF NT3, NT4, |
| | compare to | EGFR, TGFβ2, TGFβ3, | SCF R, bFGF, bNGF, |
| | untreated | VEGF, VEGF R2, | GDNF, HB, EGF, |
| | control | VEGF R3, VEGF D, | HGF, TGFα, TGFβ2, |
| | | PDGF Rα, PDGF AA, | TGFβ3, VEGF, VEGF |
| | | PDGF BB, PDGF AB, | R2, VEGF R3, VEGF |
| | | PLGF | D, PDGF AA, PDGF |
| | | | BB, PDGF AB, PLGF |

All stages of a tissue repair process are controlled by a wide variety of different growth factors, and it is known in the art that the tissue repair and healing process is benefitted by an increase production growth factors such as, e.g., insulin growth factors (IGFs) and insulin growth factor binding proteins family (IGF), nerve growth factor (NGF), epidermal growth factor (EGF) family comprising EGF, transforming growth factors α and β (TGFs), and heparin binding EGF (HB-EGF), vascular endothelial growth factor family (VEGF), platelet-derived growth factors (PDGFs) family members, fibroblast growth factors (FGFs) family members, and granulocyte-macrophage colony stimulating factor (GM-CSF). Interestingly, as indicated by the results shown in above in Table 12, upon treatment with the biophotonic PHEAA/PL-F127, a significant induction of a majority of growth factors was detected. Moreover, the effect of the biophotonic PHEAA/PL-F127-mediated induction of growth factors production was not only maintained over the time course over which the assays were taken, but also, more growth factors were detected at 72 h versus 48 h post-treatment.

In the non-IFN-γ stimulated DHF cells, no increase in growth factors production was detected, suggesting that PHEAA/PL-F127-mediated effect may be specific to the inflammatory phenotype (triggered by IFN-γ stimulation) only.

a)(iii) PHEAA/PL-F127-Mediated Effect on Inflammatory Mediators and Growth Factors Production in Wounded 3D Skin Inserts.

Observations of cellular responses in monolayer of dermal human fibroblasts upon PHEAA/PL-F127 treatment prompted us to investigate the effect mediated by this matrix on more complex cellular system, such as 3D skin. EpiDerm full thickness is an in vitro model which has both epidermis and dermis, which closely resembles to human skin. Taking advantage of these features we were able to assess the effect mediated by PHEAA/PL-F127 on the cytokines and growth factor profile in wounded skin inserts. Epidermal full thickness skin inserts were wounded using biopsy punch in order to induce acute inflammation. During the treatment PHEAA/PL-F127 was applied on the top of the skin insert. Using KLOX Thera lamp, skin inserts were illuminated with PHEAA/PL-F127 with the intensity of 14.4 J/cm² at 5 cm distance. Fresh culture media was added to the wells and 3D skin inserts were cultured at 37° C., in 5% CO₂ atmosphere. Following treatment supernatant was collected at 24 h and 72 h post-illumination and screened to detect and quantify the amount of secreted inflammatory mediators and growth factors. The results of the protein array using collected supernatants are summarized in Table 13.

TABLE 13
Summary of significant up (↑) and down-regulation (↓) observed
in inflammatory mediators and growth factors production (cytokines
in red, chemokines in blue, and growth factors in green) in wounded
3D skin inserts treated with PHEAA/PL-F127 in combination with
KLOX Multi LED light compared to untreated controls.

| | PHEAA/PL-F127 in combination with KLOX Multi LED light |
|---|---|
| | 14.4 J/cm² |
| 24 h | ↓ IL6, TNFα, TNFβ, IL1α, IL1β, IL12 p40/70, MCP1, |
| | MCP2, TARC, GROα |
| | ↑ IL3, EGF, IGF-1, ANG, VEGF |
| 72 h | ↓ IL3, IL6, IL12p40/70, TNFα, TNFβ, IL1β, IL12p40/70, |
| | IFNγ, GROα, MIP-1 Δ, TARC, PDGF-BB, TGF-β1, |
| | ↑IL8, ENA 78, EGF, IGF-1, ANG, VEGF |

Inflammatory cytokines array analysis revealed that PHEAA/PL-F127 mediates anti-inflammatory effect on wounded EpiDerm full thickness skin inserts. The pattern of up- and down-regulated mediators and growth factors resembles this one observed in monolayer of dermal human fibroblasts.

Tested radiant fluency of 14.4 J/cm² proved to reduce production of pro-inflammatory cytokines (such as IL6, TNFα, TNFβ, IL1α, and IL1β) which are hallmarks of inflammation. Along with these cytokines, the level of certain chemokines (such as MCP1, MCP2, MIP-1Δ, TARO, and GRO-α) which act as chemoattractant for the inflammatory cells to bring them to the site of inflammation, has also been significantly reduced.

Certain growth factors (such as EGF, IGF-1, ANG, and VEGF), which beneficial effect on the wound healing process has been proved, were significantly upregulated. Interestingly, the effect of PHEAA/PL-F127 illumination was maintained up to 72 h post-treatment.

EpiDerm full thickness skin system allowed us to confirm previous observations made in monolayer of dermal human fibroblasts and proved that treatment with PHEAA/PL-F127 treatment could facilitate and accelerate the resolution of inflammation

By reducing the duration of inflammation along with the level of major cytokine players, PHEAA/PL-F127 treatment could accelerate the healing process and shorten the recovery process.

Supernatant from the treated Dermal Human Fibroblast cultures described above were also screened for lactate dehydrogenase (LDH) activity in order to assess the cytotoxicity of the treatment. No significant cytotoxic effect of the treatment was observed in all PHEAA/PL-F127 illuminated skin inserts.

b)(i) PHEAA/PL-F127-Mediated Proliferation of Dermal Human Fibroblast.

Significantly increased growth factors production upon PHEAA/PL-F127 treatment correlates directly with the increased proliferation rate observed in DHF cells. These observations were made when DHF cells were illuminated with PHEAA/PL-F127 and visible blue light (KLOX Thera™ lamp). Fold increase in the proliferation potential of DHF upon treatment is summarized in Table 14.

TABLE 14
Cell proliferation expressed as fold increase
compared to untreated controls

| | | | PHEAA/PL-F127 in |
|---|---|---|---|
| Time post- | Control | KLOX Multi LED | combination with |
| treatment | (non-treated) | light only | KLOX Multi LED light |
| 24 h | 1 | 1 | 1 |
| 48 h | 1 | 1.4 | 2.82 |
| 72 h | 1 | 1.1 | 2.99 |

After 48 h the cultures were confluent with a 3-fold cell proliferation in treated samples. Proliferation assay performed on cells treated with PHEAA/PL-F127 revealed nearly 3-fold increase in the proliferation rate of DHF compare to untreated control cells. This effect was observed up to 72 h post-illumination (longer time points were not tested under these experimental settings).

These data suggested that PHEAA/PL-F127 triggers the cellular mechanism(s) responsible for accelerated cellular growth and increased proliferation potential. These observations correlate with the previous results, which demonstrated significantly increased production of variety of growth factors implicated in the proliferation process.

b)(ii) PHEAA/PL-F127-Mediated Effect on Matrix Metalloproteinases (MMPs) and Tissue Inhibitor of Matrix Metalloproteinases (TIMPs) Production in Dermal Human Fibroblast.

At 24 h post-treatment supernatant was collected and MMPs and TIMPs level was evaluated by MMP and TIMPs antibody array. The results of the array are summarized in Table 15.

TABLE 15
MMPs (in red) and TIMPs (in blue) level in PHEAA/PL-
F127 treated dermal human fibroblasts.

| | PHEAA/PL127 with KLOX Multi LED light | | |
|---|---|---|---|
| | 14.4 J/cm² | 19.5 J/cm² | 24 J/cm² |
| 24 h | No changes | ↓MMP2, MMP3 | ↑ MMP10, MMP13, |
| | observed compare | | TIMP4; |
| | to untreated | | |
| | control | | |

Performed analysis of MMPs and TIMPs level in DHF culture treated with PHEAA/PL-F127 revealed that majority of tested MMPs and TIMPs (i.e., MMP1, MMP9, MMP10, TIMP1, and TIMP2) remains unchanged and no significant increase or decrease in their production was observed at 24 h post-treatment.

The level of MMP2 (involved in collagen type IV and gelatin degradation) along with MMP3 (involved in MMP1, MMP1 and MMP9 activation and collagen type II, III and IV degradation) was decreased in DHF illuminated at 19.5 J/cm² power density.

Interestingly, at the higher power density of 24 J/cm² DHF produced increased amount of MMP10 (involved in proteoglycans and fibronectins degradation) and MMP13 (implicated in type II collagen cleavage). This was accompanied by elevated TIMP4 (involved in the regulation of the proteolytic activity of MMPs) production at 24 h post-treatment.

No significant changes in the MMPs and TIMPs level were detected in resting, non IFN-γ-stimulated fibroblast upon PHEAA/PL-F127 treatment.

c) PHEAA/PL-F127-Mediated Effect on Total Collagen Production in Dermal Human Fibroblast Culture.

Unchanged level of MMPs in PHEAA/PL-F127 treated DHF at 14.4 J/cm² as compared to untreated control cells correlates directly with the increased level of collagen proteins observed at the same dose. Collagens are crucial components of extracellular matrix involved in new tissue formation. Obtained results are summarized in Table 16.

TABLE 16
Total collagen (μg/ml) secreted by DHF upon PHEAA/PL-
F127 in combination with light treatment (14.4 J/cm).

| | | | | PHEAA/PL-F127 in |
|---|---|---|---|---|
| | | | KLOX Multi | combination with |
| | Untreated | Positive control | LED light | KLOX Multi LED |
| | Control | (Vit. C + TGFβ1) | only | light |
| 48 h | 7.5 | 12.8 | 19.9 | 45.9 |

Total collagen production analysis revealed that PHEAA/PL-F127-treated dermal human fibroblast produced and secreted 6 times more collagen then untreated control cells, suggesting that PHEAA/PL-F127 possess the ability to trigger cellular mechanism(s) which leads to increased collagen production.

It should be appreciated that the disclosure is not limited to the particular embodiments described and illustrated herein but includes all modifications and variations falling within the scope of the disclosure as defined in the appended claims.

1. A biophotonic hydrogel composition, comprising:
N-Hydroxyethyl acrylamide (HEAA), and
at least one chromophore,
wherein said chromophore does not fully photobleach after photopolymerization of the composition.

2. The biophotonic hydrogel composition of claim 1, further comprising a cross linker selected from the group consisting of poly(ethylene glycol) diacrylate (PEGDA), N,N′-methylene bis(meth)acrylamide, poly(ethylene glycol) di(meth)acrylate, poly(propylene) glycol di(meth)acrylate, glycerol di(meth)acrylate, glycerol acrylate methacrylate, trimethylolpropane di(meth) acrylate, trimethylol propane acrylate methacrylate, pentaerythritol di(meth)acrylate, glycerol tri(meth)acrylate, trimethylolpropane tri(meth)acrylate, pentaerythritol tri(meth)acrylate, pentaerythritol tetra(meth)acrylate, triallyl amine, poly(allyloxy)alkane, trialyl cyanurate, triallyl isocyanurate, triallyl phosphate, poly(ethylene glycol) diglycidyl ether, propylene glycol diglycidyl ether, glycerol diglycidyl ether, glycerol triglycidyl ether, 2,4-toluylene diisocyanate, hexamethylene diisocyanate, polyoxazoline compounds, N-methylol(meth)acryl amide, and glycidyl(meth)acrylate.

3. (canceled)

4. The biophotonic hydrogel composition of claim 1, further comprising an initiator.

5. (canceled)

6. The biophotonic hydrogel composition of claim 1, further comprising a catalyst selected from the group consisting of 1-vinyl-2 pyrrolidinone (NVP) and polyvinyl pyrrolidone (PVP).

7-9. (canceled)

10. The biophotonic hydrogel composition of claim 1, further comprising a surfactant selected from the group consisting of Poloxamer 407 and cetrimonium bromide (CTAB).

11. The biophotonic hydrogel composition of claim 10, wherein the surfactant is Poloxamer 407.

12. The biophotonic hydrogel composition of claim 11, wherein the content of Poloxamer 407 in the composition is in an amount of from about 5 wt % to about 50 wt %.

13-14. (canceled)

15. The biophotonic hydrogel composition of claim 1, further comprising an agent that increases the mechanical strength of the composition, and wherein the agent is a surfactant, silicon dioxide (SiO2), bentonite, or a combination thereof.

16. (canceled)

17. The biophotonic hydrogel composition of claim 1, further comprising a thickening agent selected from the group consisting of gelatin, hydroxyethyl cellulose (HEC), carboxymethyl cellulose (CMC), xanthan gum, guar gum, and starch.

18. (canceled)

19. The biophotonic hydrogel composition of claim 1, further comprising an antimicrobial agent.

20. The biophotonic hydrogel composition of claim 1, wherein the content of HEAA in the composition is in an amount of from about 5 wt % to about 50 wt % or of from about 15 wt % to about 25 wt %.

21-23. (canceled)

24. The biophotonic hydrogel composition of claim 1, wherein the chromophore is selected from the group consisting of a xanthene dye, a chlorophyll dye, and an azo dye.

25. The biophotonic hydrogel composition of claim 24, wherein the xanthene dye is selected from the group consisting of Eosin Y, Erythrosine B, Fluorescein, Rose Bengal, and Phloxin B.

26. (canceled)

27. The biophotonic hydrogel composition of claim 1, wherein the chromophore is present in an amount of from about 0.005 wt % to about 5 wt % or in an amount of from about 0.005 wt % to about 0.1 wt %.

28. (canceled)

29. The biophotonic hydrogel composition of claim 25, further comprising Fluorescein, Erythrosine B, Rose Bengal, Phloxin B, or combinations thereof.

30. A method for promoting wound healing comprising:
applying a biophotonic hydrogel composition over a wound, wherein the biophotonic hydrogel composition comprises N-Hydroxyethyl acrylamide (HEAA) and at least one chromophore; and
illuminating said biophotonic hydrogel composition with light having a wavelength that is absorbed by said chromophore;
wherein said method promotes wound healing.

31-35. (canceled)

36. A method for biophotonic skin treatment comprising:
applying a biophotonic hydrogel composition over a skin, wherein the hydrogel composition comprises N-Hydroxyethyl acrylamide (HEAA), and at least one chromophore; and
illuminating said biophotonic hydrogel composition with light having a wavelength that is absorbed by the at least one chromophore; and
wherein said method promotes treatment of said skin.

37. (canceled)

38. The method of claim 30 or 36, wherein the chromophore is a xanthene dye selected from the group consisting of Eosin Y, Erythrosine B, Fluorescein, Rose Bengal, and Phloxin B.

39. (canceled)

40. A kit for preparation of the biophotonic hydrogel composition of claim 1, comprising:
the N-Hydroxyethyl acrylamide (HEAA);
the at least one chromophore; and
at least one container.

41-42. (canceled)

43. The biophotonic hydrogel composition of claim 19, wherein the antimicrobial agent is selected from the group consisting of hydrogen peroxide, benzoyl peroxide, and urea peroxide.

44. The method of claim 30, wherein the method comprises treating or preventing scarring.

45. The method of claim 36, wherein the skin treatment comprises a skin disorder selected from the group consisting of acne, eczema, psoriasis, and dermatitis.

46. The method of claim 36, wherein the skin treatment comprises promoting skin rejuvenation.
